(19) United States
(12) Patent Application Publication
(10) Pub. No.: US 2012/0253470 A1
(43) Pub. Date: Oct. 4, 2012

(54) COMPOSITIONS FOR BONE TISSUE REPAIR AND USES THEREOF

(75) Inventors: Kevin A. Guze, Lexington, MA (US); Vinicius Souza Rodrigues, Madison Heights, MI (US)

(73) Assignee: PRESIDENT AND FELLOWS OF HARVARD COLLEGE, Boston, MA (US)

(21) Appl. No.: 13/435,259

(22) Filed: Mar. 30, 2012

Related U.S. Application Data
(60) Provisional application No. 61/469,248, filed on Mar. 30, 2011.

(51) Int. Cl. A61F 2/28

(52) U.S. Cl. 623/23.51

(57) ABSTRACT

The technology described herein relates to compositions for promoting bone growth or regeneration. The compositions described herein can comprise bioactive agents and scaffold materials.

### CROSS REFERENCE TO RELATED APPLICATIONS

This application claims benefit under 35 U.S.C. §119(e) of U.S. Provisional Application No. 61/469,248 filed Mar. 30, 2011, the contents of which are incorporated herein by reference in its entirety.

### TECHNOLOGICAL FIELD

Embodiments of the technology described herein relate to tissue repair and regeneration. Specifically, embodiments relate to the healing, repair, and regeneration of hard and soft tissue defects. In particular, the embodiments herein relate to compositions for promoting and/or enhancing the repair and regeneration of tissues with defects.

### BACKGROUND

When a tissue (e.g., bones, skin, etc) sustains a small amount of damage, for example, a minor bone fracture, the tissues involved are able to regenerate, growing enough new material to seal the fissure and replace any dead or damaged tissue. However, if a tissue is more extensively damaged, such as a major bone fracture due to disease or trauma, repair becomes more problematic. Most tissues will not readily grow into a newly empty space which now lacks an appropriate tissue infrastructure. While this is normally an important attribute that maintains correct anatomy it limits the extent of healing; growth of new tissue will be slow or may not occur at all, leading to a permanent injury, defects or deformities.

In repairing large tissue defects, both new tissue growth and vascularization must be encouraged in order to promote tissue repair and regeneration. A variety of materials are used. For example, donor tissue grafts, synthetic tissue grafts, growth factors, and stem cells.

Technologies have been developed to aid in overcoming the natural obstacles in healing large (critical size) defects. Grafts, for hard or soft tissue, provide healthy tissue and a scaffold that encourages existing tissues to expand into the area needing repair. Recent developments in tissue engineering have provided graft materials which are seeded with cells particularly disposed for rapid growth, or which contain agents that will stimulate growth of graft and/or host cells. Peptide agents have shown particular potential and use of single agents has had a demonstrable benefit, but more advanced compositions that utilize the natural interplay of growth factors are called for. However, the use of growth factors has been complicated by dosing issues caused by short half-lives as growth factors. Additionally, donor tissue grafts is limited in supply and carries significant costs such as donor site morbidity, immune reactions, and inconsistency in the grafts. Innovative techniques that address these limitations can lessen the need for graft tissues, speed recovery times, and lower both risks and costs for the subject.

### SUMMARY

Embodiments of the technology described herein are based on the discovery that a fabricated composition comprising pro-angiogenic factors, demineralized bone particles and a scaffold material greatly enhanced and promoted bone tissue repair, particularly when the repair needed covers a large area and/or volume, e.g., larger than 2 mm in length and/or 0.1 cm² in area. The porous fabricated composition comprised poly(lactic-co-glycolic acid) (PLGA) microspheres that encapsulated platelet-derived growth factor (PDGF) within, vascular endothelial growth factor (VEGF), prominin-1 peptide #237 (peptide #237), and demineralized bone particles that were pressure compressed together. The porous fabricated composition provided sustained-release of bioactive agents at the repair site and enhanced the repair process. The demineralized bone particles provide the initial tissue infrastructure for regeneration to take place.

Accordingly, in one embodiment, provided herein is a composition for promoting bone growth and/or bone regeneration, the composition comprises at least one bioactive agent, a first scaffold material and at least one other scaffold material. In one embodiment, the at least one other scaffold material forms a microsphere.

In another embodiment, provided herein is a composition for promoting bone growth and/or bone regeneration, the composition comprises a microsphere, at least one bioactive agent and at least one scaffold material.

In another embodiment, provided herein is a method of promoting bone growth and/or bone regeneration in a hard tissue or an oral, the method comprising contacting the tissue with a composition described herein. In another embodiment, the method comprises implanting the composition into the tissue. In another embodiment, the method comprises shaping the composition to fit into the tissue and then implanting the composition into the tissue

In another embodiment, provided herein is a method of treating or repairing a hard tissue defect or an oral tissue defect in a subject, the method comprising contacting the defect with a composition described herein. In another embodiment, the method comprises implanting the composition into the tissue. In another embodiment, the method comprises shaping the composition to fit into the tissue, taking the shape of the defect, and then implanting the composition into the tissue.

In another embodiment, provided herein is a method of treating or repairing a hard tissue defect or an oral tissue defect in a subject, the method comprising selecting a subject having the defect and contacting the defect in the subject with a composition described herein. In another embodiment, the method comprises implanting the composition into the tissue. In another embodiment, the method comprises shaping the composition to fit into the tissue, taking the shape of the defect, and then implanting the composition into the tissue.

In one embodiment, the at least one bioactive agent is an agent that promotes angiogenesis. For example, vascular endothelial growth factor (VEGF), platelet-derived growth factor (PDGF), fibroblast growth factor (FGF) and prominin-1 peptide (peptide #237 aka prom-1 #237).

In another embodiment, the at least one bioactive agent is an agent that promotes bone growth. For example, bone morphogenetic protein 2 (BMP2), BMP4 and BMP7.

In one embodiment, the at least one bioactive agent is a growth factor. For example, epidermal growth factor (EGF), transforming growth factor beta (TGF-β), migration-stimulating factor, and insulin-like growth factor (IGF).

In some embodiments, at least one bioactive agent is encapsulated within a scaffold material, For example, encapsulation within a microsphere. The microspheres facilitate sustained release of the bioactive agent at the contacting site or implant site.

In one embodiment, the first scaffold material is a bone fragment or a bone-derived fragment. For example, demineralized bone particles, mineralized bone particles, demineralized freeze-dried bone particles, mineralized freeze-dried bone particles, ceramic particles, cancellous chips and collagen.

In one embodiment of the composition, the at least one bioactive agent, the first scaffold material and the at least one other scaffold material are solidified together into a fabricated structure having a defined shape and size.

In one embodiment of the composition further comprising a solidifying agent for solidifying the at least one bioactive agent, the first scaffold material and the at least one other scaffold material into a fabricated structure having a defined shape and size.

In one embodiment, the composition comprises a PLGA microsphere, VEGF, PDGF, peptide #237 and demineralized bone particles, wherein the PLGA microsphere encapsulate PDGF within and is coated with VEGF externally. In another embodiment, the composition consists of a PLGA microsphere, VEGF, PDGF, peptide #237 and demineralized bone particles, wherein the PLGA microsphere encapsulate PDGF within and is coated with VEGF externally. In another embodiment, the composition consists essentially of a PLGA microsphere, VEGF, PDGF, peptide #237 and demineralized bone particles, wherein the PLGA microsphere encapsulate PDGF within and is coated with VEGF externally.

In one embodiment, the composition comprises a PLGA microsphere, PDGF, peptide #237 and demineralized bone particles, wherein the PLGA microsphere encapsulates PDGF within. In another embodiment, the composition consists of a PLGA microsphere, PDGF, peptide #237 and demineralized bone particles, wherein the PLGA microsphere encapsulates PDGF within. In another embodiment, the composition consists essentially of a PLGA microsphere, PDGF, peptide #237 and demineralized bone particles, wherein the PLGA microsphere encapsulates PDGF within.

In one embodiment, the composition comprises a PLGA microsphere, VEGF, peptide #237 and demineralized bone particles, wherein the PLGA microsphere is coated with VEGF externally. In another embodiment, the composition consists of a PLGA microsphere, VEGF, peptide #237 and demineralized bone particles, wherein the PLGA microsphere is coated with VEGF externally. In another embodiment, the composition consists essentially of a PLGA microsphere, VEGF, peptide #237 and demineralized bone particles, wherein the PLGA microsphere is coated with VEGF externally.

In one embodiment, the composition comprises a PLGA microsphere, peptide #237 and demineralized bone particles. In another embodiment, the composition consists of a PLGA microsphere, peptide #237 and demineralized bone particles. In another embodiment, the composition consists essentially of a PLGA microsphere, peptide #237 and demineralized bone particles. It is also contemplated that the peptide #237 can be encapsulated within the PLGA microsphere and/or coats the PLGA microsphere externally.

In one embodiment, the composition comprises PLGA, VEGF, PDGF, peptide #237 and demineralized bone particles. In another embodiment, the composition consists of PLGA, VEGF, PDGF, peptide #237 and demineralized bone particles. In another embodiment, the composition consists essentially of PLGA, VEGF, PDGF, peptide #237 and demineralized bone particles.

In one embodiment, the composition comprises PLGA, PDGF, peptide #237 and demineralized bone particles. In another embodiment, the composition consists of PLGA, PDGF, peptide #237 and demineralized bone particles. In another embodiment, the composition consists essentially of PLGA, PDGF, peptide #237 and demineralized bone particles.

In one embodiment, the composition comprises PLGA, VEGF, peptide #237 and demineralized bone particles. In another embodiment, the composition consists of PLGA, VEGF, peptide #237 and demineralized bone particles. In another embodiment, the composition consists essentially of PLGA, VEGF, peptide #237 and demineralized bone particles.

In one embodiment, the composition comprises PLGA, peptide #237 and demineralized bone particles. In another embodiment, the composition consists of PLGA, peptide #237 and demineralized bone particles. In another embodiment, the composition consists essentially of PLGA, peptide #237 and demineralized bone particles.

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein, “composition” refers to an injectate, substance or a combination of substances which can be delivered into a tissue or an organ. In one embodiment, the composition is in a form of a fabricated structure such as a gel-like extracellular matrix or a biocompatible scaffold.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used herein, “bioactive agents” or “bioactive materials” refer to naturally occurring or synthetic molecules or compounds that are found in organic tissues needing repair, regeneration or new/additional growth, the molecules or compounds are those naturally needed for the repair, regeneration or growth process. The synthetic molecules or compounds mimic those of the naturally occurring synthetic molecules or compounds in functions. For example, growth factors and differentiation factors. Peptides or recombinant vascular endothelial growth factor (VEGF) that can stimulate angiogenesis. “Bioactive agents” also refer to artificially synthesized materials, molecules or compounds that have a biological effect in living tissues in general, e.g., promoting cellular growth process. Also included within the scope of “bioactive agents” are derivatives, isoforms, variants, homologs, and peptides of these compounds.

As used herein, a “scaffold” is an artificial fabricated structure, platform or framework capable of supporting three-dimensional tissue formation. For example, a scaffold allows the ingrowth of living host tissue, e.g., cells and extracellular matrix etc., within or upon the surface of the framework. A scaffold can contain bioactive agents and also releases the bioactive agents to the environment where the scaffold in placed. In this embodiment, the scaffold an artificial fabricated structure for the delivery of bioactive agents. The scaffold can be a temporary structure or a permanent structure.

The term “biodegradable” includes that all or parts of the scaffold material can degrade over time by the action of enzymes, by hydrolytic action and/or by other similar mechanisms in the human body. In various embodiments, “biodegradable” includes that the scaffold material (e.g., microparticle, microsphere, gel, etc.) can break down or degrade within the body to non-toxic components after or while a bioactive agent has been or is being released. By “bioerodible” it is meant that the scaffold material will erode or degrade over time due, at least in part, to contact with substances found in the surrounding tissue, fluids or by cellular action. By “bioabsorbable” it is meant that the scaffold material will be broken down and absorbed within the human body, for example, by a cell or tissue. “Biocompatible” means that the scaffold material will not cause substantial tissue irritation or necrosis at the target tissue site.

As used herein, the term “pro-angiogenic activity” refers to the stimulation or enhancement of angiogenesis and/or endothelial cell proliferation.

The term “angiogenesis”, as used herein refers to the sprouting of blood vessels from pre-existing blood vessels, characterized by endothelial cell proliferation and the proliferation and migration of tube forming cells. Angiogenesis is necessary process, for example, in wound healing, and new tissue growth.

As used herein, “neovascularization” refers to the formation of functional vascular networks that may be perfused by blood or blood components in tissues. Neovascularization includes angiogenesis, sprouting angiogenesis, therapeutic angiogenesis and vasculogenesis.

As used herein, the term “vasculogenesis” refers to the formation of new blood vessels when there are no pre-existing ones. Blood vessel formation occurring by a de novo process where endothelial progenitor cells (EPCs) and mesenchymal progenitor cells (MPCs) migrate, assemble and differentiate in response to local cues (such as growth factors and extracellular matrices) to form new blood vessels.

As used herein, the term “progenitor” cell refers to an immature or undifferentiated cell, typically found in post-natal animals. Progenitor cells can be unipotent or multipotent. As used herein, progenitor cells refers to either EPCs or MPCs, or both EPCs and MPCs.

As used herein, the term “pro-osteogenic properties” refers to properties that stimulate or enhance the formation or development of bone and associated process such as biosynthesis of type I collagen.

As used herein, the word “repair” means the natural replacement of worn, torn or broken components with newly synthesized components, e.g., new, healthy tissue. The word “healing”, as used herein, means the returning of torn and broken organs and tissues (wounds) to wholeness. As used herein, the terms “healing”, “repair”, and “regeneration” are used interchangeably. The process of “healing”, “repair”, and “regeneration” require, in part, angiogenesis and revascularization of the tissue.

As used herein, the terms “tissue regeneration” and “tissue repair” are related terms and used interchangeably.

As used herein, the terms “defect” or “tissue defect” are used interchangeably to refer to any medical abnormality of a tissue, including, but not limited to, a damaged tissue, a deficient tissue, a degraded tissue, a traumatized tissue. In one embodiment, any abnormality of a tissue that may be regenerated, repaired, or healed is included in the term “tissue defect”.

The term “subject” refers to an animal, particularly a human, to whom it is desirable to administer a composition described herein. The term “subject” as used herein also refers to human and non-human animals. The term “non-human animals” includes all vertebrates, e.g., mammals, such as non-human primates, (particularly higher primates), sheep, dog, rodent (e.g. mouse or rat), guinea pig, goat, pig, cat, rabbits, cows, and any domestic animal or pet, as well as non-mammals such as chickens, amphibians, reptiles etc. In one embodiment, the subject is human.

As used herein, the term “porogen” generally refers to those sacrificial materials known in the art that generate or form pores within a scaffold as removal of the porogen. The porogen forms domains or discrete regions in the scaffold or scaffold material, which upon removal of the porogen, form pores or voids.

The term “sustained release” (e.g., extended release or controlled release) is used herein to refer to one or more bioactive agents(s) and/or scaffold material that is introduced into the body of a human or other mammal and continuously releases a stream of one or more therapeutic agents over a predetermined time period and at a therapeutic level sufficient to achieve a desired therapeutic effect throughout the predetermined time period. Reference to a continuous release stream is intended to encompass release that occurs as the result of biodegradation in vivo of a matrix, or as the result of metabolic transformation or dissolution of the therapeutic agent(s) or conjugates of therapeutic agent(s).

### BRIEF DESCRIPTION OF THE DRAWINGS

FIGS. 1A-1B depict that prominin-1 #237 promotes long bone regeneration.

FIG. 2 depicts repair of critical size defects in mouse calvaria.

FIG. 3 depicts the design of the mouse calvaria defect experiment in vivo.

FIG. 4 depicts the injection and dosage regime of the composition for the various groups of mice in the calvaria defect experiment in vivo.

FIG. 5 depicts the effects of PDGF, VEGF, and/or prom-1 #237 (peptide) on bone healing in the absence of demineralized freeze-dried bone graft (DFDBA) in the calvaria defect experiment in vivo.

FIG. 6 depicts the effects of PDGF, VEGF, and/or prom-1 #237 (peptide) on bone healing in the presence of demineralized freeze-dried bone graft (DFDBA) in the calvaria defect experiment in vivo.

### DETAILED DESCRIPTION

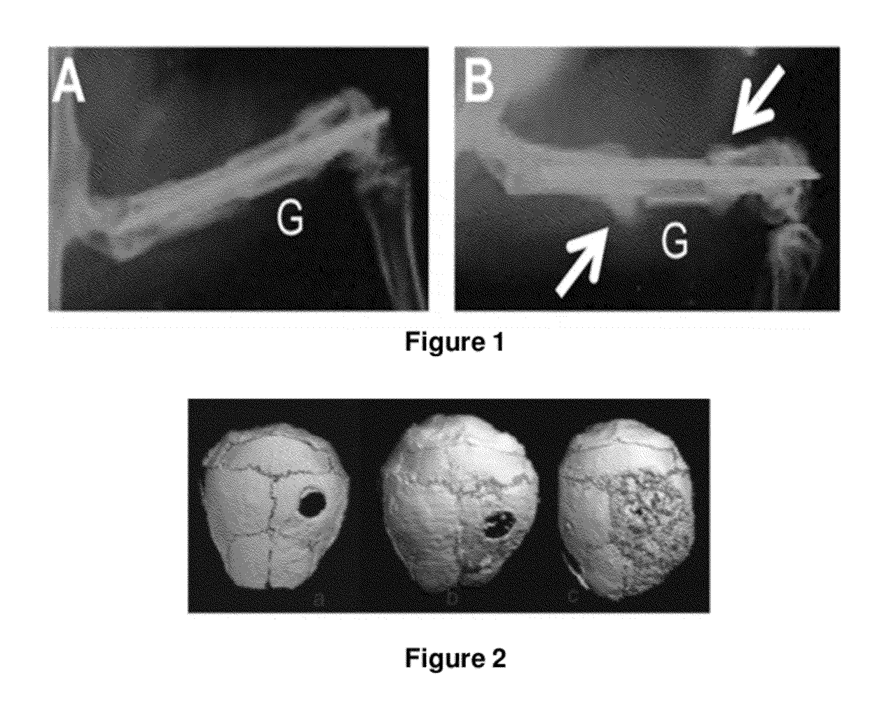

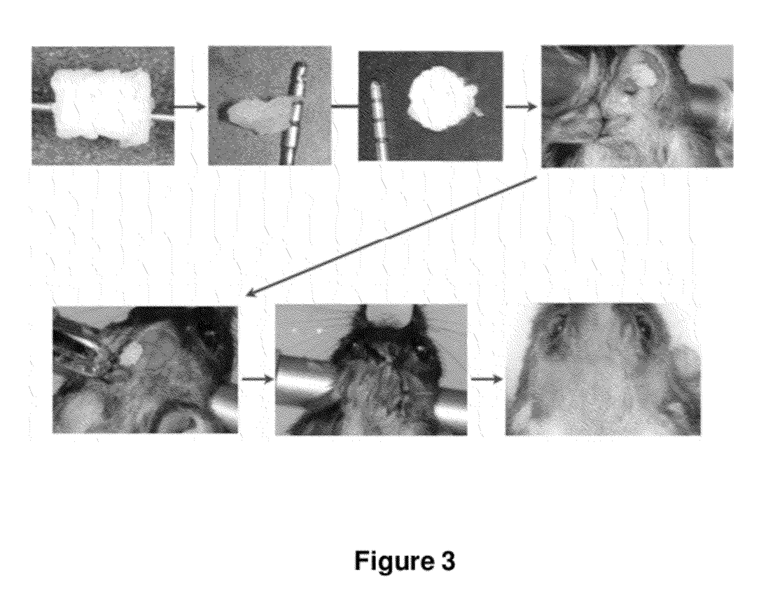

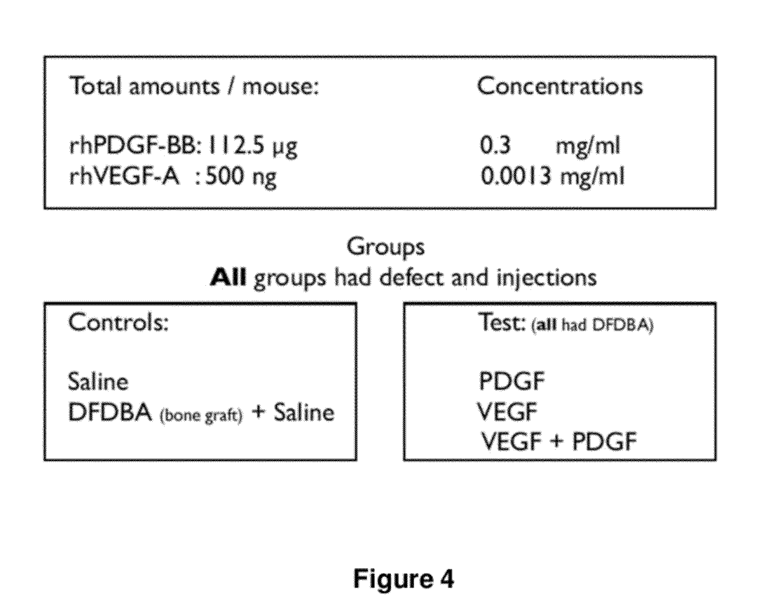

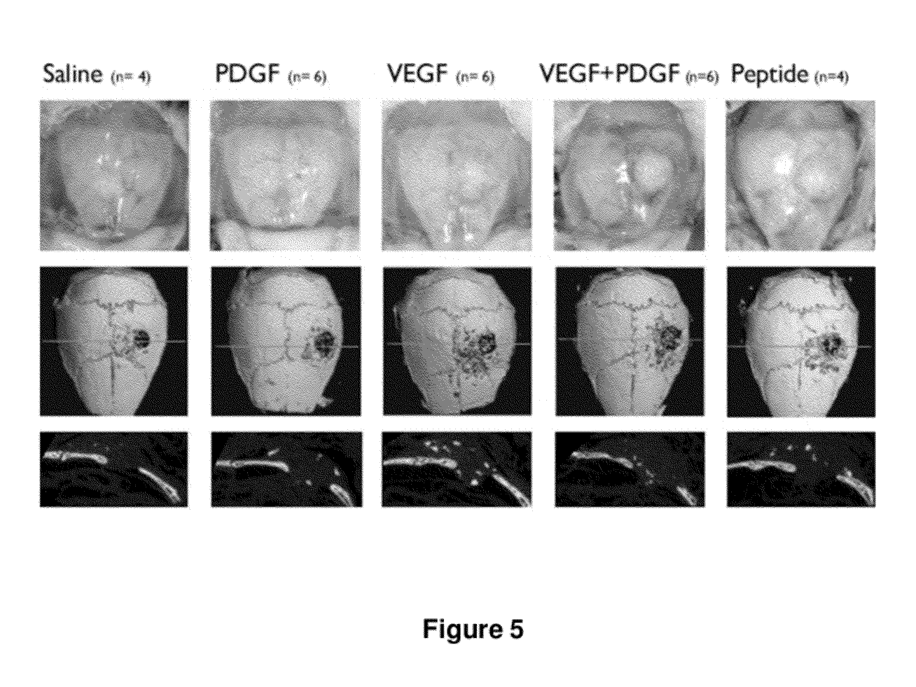

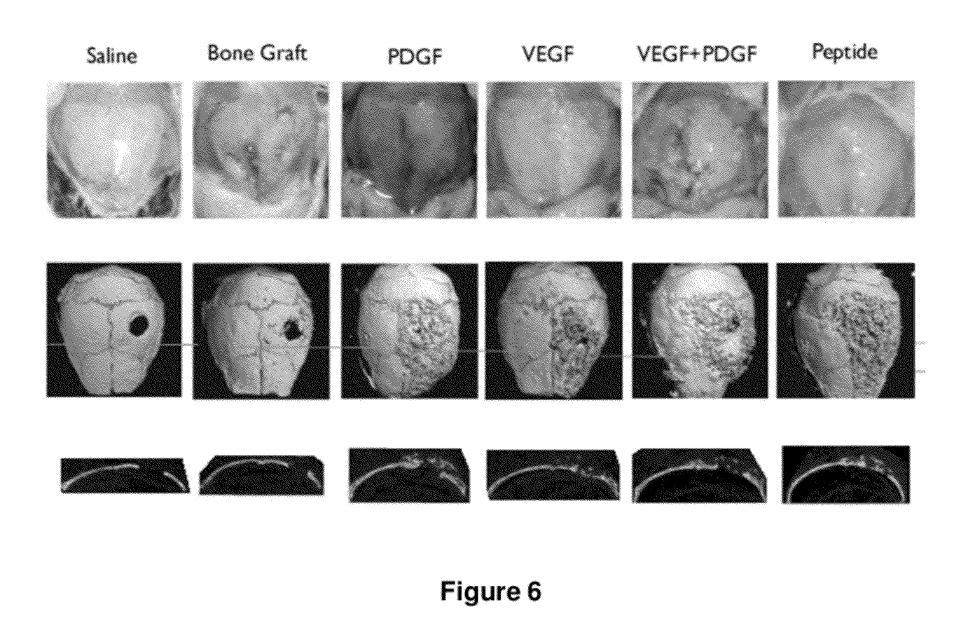

Embodiments of the technology described herein are based on the discovery that a fabricated composition comprising pro-angiogenic factors, demineralized bone particles and a scaffold material greatly enhanced and promoted bone tissue repair, particularly when the repair needed covers a large area and/or volume, e.g., larger than 2 mm in length and 0.1 cm² in area. The inventors mixed poly(lactic-co-glycolic acid) (PLGA) microspheres that encapsulated platelet-derived growth factor (PDGF) within with vascular endothelial growth factor (VEGF), prominin-1 peptide #237 (peptide #237), and demineralized bone particles together and compressed the mixture to form a fabricated composition. The fabricated composition is sized and shaped to fit into the space of a tissue needing repair, i.e. the wound, e.g., missing length of the long leg bone femur (see FIG. 1) or a missing area of a skull bone (see FIG. 2). The fabricated composition is then placed into the repair area, filing the void space. In this configuration at the repair or wound site, the fabricated composition provides local sustained concentrations of pro-angiogenic factors that promote cell migration of non-differentiated cells and neovascularization of the new tissue, etc, as well as provide the scaffold material for initiating new bone growth.

Such fabricated compositions can be highly useful for promoting bone growth and/or bone regeneration in a tissue, e.g., for repairing defects or wounds in hard tissues or oral tissues. For example, lost or damaged bone structures in any part of the body due to diseases, trauma, ordinary wear and tear with time etc. The fabricated compositions can also be highly useful for promoting bone growth and/or bone regeneration in a tissue where additional bone growth is desired, e.g., in spinal fusion, spinal disc decompression, and sinus lift.

By administering a number of bioactive agents in a controlled-release manner and simultaneously provided suitable scaffold material for cell growth by way of the demineralized bone particles, healing of critical size defects is significantly improved. In addition to promoting faster and more complete healing of tissue defects, the fabricated compositions lessens the need for graft tissue, which is limited in supply and carries significant costs such as donor site morbidity, immune reactions, and inconsistency in the end product. The fabricated composition thus speeds recovery times, improves subject outcomes, and lowers both risks and costs for the subject by enhancing tissue healing, repair, and regeneration.

Accordingly, in one embodiment, provided herein is a composition for promoting bone growth and/or bone regeneration, the composition comprises at least one bioactive agent, a first scaffold material and at least one other scaffold material.

In one embodiment of the composition, the at least one other scaffold material forms a microsphere.

Accordingly, in one embodiment, provided herein is a composition for promoting bone growth and/or bone regeneration, the composition comprises a microsphere, at least one bioactive agent and at least one scaffold material.

In another embodiment, provided herein is a method of promoting bone growth and/or bone regeneration in a hard tissue or an oral, the method comprising contacting the tissue with a composition described herein.

In another embodiment, provided herein is a method of treating or repairing a hard tissue defect or an oral tissue defect in a subject, the method comprising contacting the defect with a composition described herein.

In another embodiment, provided herein is a method of treating or repairing a hard tissue defect or an oral tissue defect in a subject, the method comprising selecting a subject having the defect and contacting the defect in the subject with a composition described herein.

In one embodiment, the at least one bioactive agent is an agent that promotes angiogenesis. Numerous growth factors are known in the art to induce and/or enhance angiogenesis and neovascularization. These factors are often termed “pro-angiogenic factors”. In one embodiment, the at least one bioactive agent selected from the group consisting of vascular endothelial growth factor (VEGF), platelet-derived growth factor (PDGF), fibroblast growth factor (FGF) and prominin-1 peptide (peptide #237).

In one embodiment, the at least one bioactive agent is an agent that promotes bone growth. Numerous bone growth factors are known in the art to induce bone growth, e.g., lactoferrin, bone morphogenetic protein 2 (BMP2), BMP4 and BMP7. In one embodiment, the at least one bioactive agent selected from the group consisting of lactoferrin, BMP2, BMP4, BMP 6, BMP7, and BMP9.

In one embodiment, the at least one bioactive agent is a growth factor. A growth factor is a naturally occurring substance or a synthetic form thereof capable of stimulating cellular growth, proliferation and cellular differentiation. In some embodiment, the growth factor is a protein or a steroid hormone. Numerous growth factors are known in the art, e.g., epidermal growth factor (EGF), transforming growth factor beta (TGF-β), migration-stimulating factor, and insulin-like growth factor (IGF).

In one embodiment, the composition comprises 0.5 ng to 5 mg of the bioactive agent. In other embodiments, the amount of bioactive agent in the composition is in the range of 5 ng to 0.1 mg; 1 ng to 0.2 mg; 10 ng to 100 μg, 50 ng to 50 μg, 25 ng to 10 μg, 10 μg to 50 μg, 1 μg to 50 μg, 1 μg to 25 μg, 10 μg to 100 μg, 5 μg to 10 μg, 5 μg to 25 μg, and 10 μg to 50 μg. In other embodiments, the amount of bioactive agent in the composition is 1 ng, 10 ng, 50 ng, 75 ng, 100 ng, 150 ng, 0.25 μg, 1 μg, 5 μg, 10 μg, 30 μg, 50 μg, 75 μg, 100 μg, 0.2 mg, 0.5 mg, 0.75 mg, 1 mg, 1.5 mg or 2.5 mg.

In one embodiment, the at least one scaffold material of the compositions provides the initial infrastructure around which the new bone growth can take place. In one embodiment, the first scaffold material of the compositions provides the initial infrastructure around which the new bone growth takes place. In one embodiment, the initial infrastructure around which the new bone growth can takes place is a bone fragment or a bone-derived fragment. In one embodiment, the first scaffold material is a bone fragment or a bone-derived fragment. In one embodiment, the bone fragment is a demineralized bone particle. The bone fragment or the bone-derived fragment can be obtained from a human or any other animal having a calcified endoskeleton, e.g., a cow or a pig. In other embodiments, the bone fragment or the bone-derived fragment is obtained from any other animal having a calcified exoskeleton, e.g., mussels and crustaceans.

In another embodiment, the first scaffold material is not a bone fragment or a bone-derived fragment. In one embodiment, the first scaffold material is a cartilage fragment or a cartilage-derived fragment. In one embodiment, the first scaffold material is a synthetic cartilage material, e.g., CARTIVA™ MTP1 cartilage. In another embodiment, the first scaffold material is any known scaffold material other than a bone fragment or a bone-derived fragment, e.g., ceramic particles, calcium phosphate, HYDROSET bone substitute from Stryker® and collagen.

In one embodiment, the first scaffold material is selected from the group consisting of demineralized bone particles, mineralized bone particles, demineralized freeze-dried bone particles, mineralized freeze-dried bone particles, ceramic particles, cancellous chips and collagen.

In one embodiment, the scaffold material of the composition is selected from but not limited to the group consisting of glycosaminoglycan, silk, fibrin, MATRIGEL™, poly-ethyleneglycol (PEG), polyhydroxy ethyl methacrylate, polyvinyl alcohol, polyacrylamide, poly(N-vinyl pyrolidone), poly glycolic acid (PGA), poly lactic-co-glycolic acid (PLGA), poly e-carpolactone (PCL), polyethylene oxide, poly propylene fumarate (PPF), poly acrylic acid (PAA), hydrolysed polyacrylonitrile, polymethacrylic acid, polyethylene amine, alginic acid, pectinic acid, carboxy methyl cellulose, hyaluronic acid, heparin, heparin sulfate, chitosan, carboxymethyl chitosan, chitin, pullulan, gellan, xanthan, collagen, gelatin, carboxymethyl starch, carboxymethyl dextran, chondroitin sulfate, cationic guar, cationic starch as well as salts and esters thereof.

In other embodiments, the scaffold material of the composition is one or a mixture of collagen, alginic acid, pectinic acid, carboxymethyl cellulose, hyaluronic acid, chitosan, polyvinyl alcohol and salts and esters thereof. Preferred anionic polymers are alginic or pectinic acid; preferred cationic polymers include chitosan, cationic guar, cationic starch and polyethylene amine. Other preferred polymers include esters of alginic, pectinic or hyaluronic acid and C2 to C4 polyalkylene glycols, e.g. propylene glycol, as well as blends containing 1 to 99 wt % of alginic, pectinic or hyaluronic acid with 99 to 1 wt % polyacrylic acid, polymethacrylic acid or polyvinylalcohol. Preferred blends comprise alginic acid and polyvinylalcohol. Examples of mixtures include but are not limited to a blend of polyvinyl alcohol (PVA) and sodium alginate and propyleneglycol alginate.

In one embodiment, the composition comprises at least three different scaffold materials that are known in the art and described herein. In other embodiments, the composition comprises two scaffold materials, three scaffold materials, or four scaffold materials. In another embodiment, the composition comprises up to ten scaffold materials. In certain embodiments, the composition comprises two, three, four, five, six, seven, eight, nine or ten scaffold materials. In other embodiments, the composition consists essentially of two, three, four, five, six, seven, eight, nine or ten scaffold materials.

In one embodiment, the composition consists essentially of at least three different scaffold materials that are known in the art and described herein. In other embodiments, the composition consists essentially of two scaffold materials, three scaffold materials, or four scaffold materials. In another embodiment, the composition consists essentially of up to ten scaffold materials.

In one embodiment of the composition, at least one of the scaffold materials provides the initial infrastructure around which the new bone growth can take place. In one embodiment of the composition, at least one of the scaffold materials is a bone fragment or a bone-derived fragment. In one embodiment of the composition, at least one of the scaffold materials is a demineralized bone particle. In one embodiment of the composition, at least one of the scaffold materials is demineralized freeze-dried bone graft (DFDBA). In one embodiment of the composition, at least one of the scaffold materials is a freeze-dried bone graft (FDBA).

In one embodiment of the composition, the scaffold material is PLGA. In one embodiment of the composition, the at least one scaffold material is PLGA. In one embodiment of the composition, at least one of the scaffold materials is PLGA. In one embodiment of the composition, the first scaffold material is PLGA.

In one embodiment of the composition, the scaffold material is collagen. In one embodiment of the composition, the at least one scaffold material is collagen. In one embodiment of the composition, at least one of the scaffold materials is collagen. In one embodiment of the composition, the first scaffold material is collagen.

In one embodiment, the composition comprises PLGA, VEGF, PDGF, peptide #237 and demineralized bone particles. In another embodiment, the composition consists of PLGA, VEGF, PDGF, peptide #237 and demineralized bone particles. In another embodiment, the composition consists essentially of PLGA, VEGF, PDGF, peptide #237 and demineralized bone particles.

In one embodiment, the composition comprises PLGA, PDGF, peptide #237 and demineralized bone particles. In another embodiment, the composition consists of PLGA, PDGF, peptide #237 and demineralized bone particles. In another embodiment, the composition consists essentially of PLGA, PDGF, peptide #237 and demineralized bone particles.

In one embodiment, the composition comprises PLGA, VEGF, peptide #237 and demineralized bone particles. In another embodiment, the composition consists of PLGA, VEGF, peptide #237 and demineralized bone particles. In another embodiment, the composition consists essentially of PLGA, VEGF, peptide #237 and demineralized bone particles.

In one embodiment, the composition comprises PLGA, peptide #237 and demineralized bone particles. In another embodiment, the composition consists of PLGA, peptide #237 and demineralized bone particles. In another embodiment, the composition consists essentially of PLGA, peptide #237 and demineralized bone particles.

In one embodiment, the composition comprises collagen, VEGF, PDGF, peptide #237 and demineralized bone particles. In another embodiment, the composition consists of collagen, VEGF, PDGF, peptide #237 and demineralized bone particles. In another embodiment, the composition consists essentially of collagen, VEGF, PDGF, peptide #237 and demineralized bone particles.

In one embodiment, the composition comprises collagen, PDGF, peptide #237 and demineralized bone particles. In another embodiment, the composition consists of collagen, PDGF, peptide #237 and demineralized bone particles. In another embodiment, the composition consists essentially of collagen, PDGF, peptide #237 and demineralized bone particles.

In one embodiment, the composition comprises collagen, VEGF, peptide #237 and demineralized bone particles. In another embodiment, the composition consists of collagen, VEGF, peptide #237 and demineralized bone particles. In another embodiment, the composition consists essentially of collagen, VEGF, peptide #237 and demineralized bone particles.

In one embodiment, the composition comprises collagen, peptide #237 and demineralized bone particles. In another embodiment, the composition consists of collagen, peptide #237 and demineralized bone particles. In another embodiment, the composition consists essentially of collagen, peptide #237 and demineralized bone particles.

In one embodiment, the composition comprises at least one scaffold material, VEGF, PDGF, peptide #237 and demineralized bone particles. In another embodiment, the composition consists of at least one scaffold material, VEGF, PDGF, peptide #237 and demineralized bone particles. In another embodiment, the composition consists essentially of at least one scaffold material, VEGF, PDGF, peptide #237 and demineralized bone particles.

In one embodiment, the composition comprises at least one scaffold material, PDGF, peptide #237 and demineralized bone particles. In another embodiment, the composition consists of at least one scaffold material, PDGF, peptide #237 and demineralized bone particles. In another embodiment, the composition consists essentially of at least one scaffold material, PDGF, peptide #237 and demineralized bone particles.

In one embodiment, the composition comprises at least one scaffold material, VEGF, peptide #237 and demineralized bone particles. In another embodiment, the composition consists of at least one scaffold material, VEGF, peptide #237 and demineralized bone particles. In another embodiment, the composition consists essentially of at least one scaffold material, VEGF, peptide #237 and demineralized bone particles.

In one embodiment, the composition comprises at least one scaffold material, peptide #237 and demineralized bone particles. In another embodiment, the composition consists of at least one scaffold material, peptide #237 and demineralized bone particles. In another embodiment, the composition consists essentially of at least one scaffold material, peptide #237 and demineralized bone particles.

In one embodiment, the composition comprises at least one scaffold material, at least one bioactive agent, peptide #237 and demineralized bone particles. In another embodiment, the composition consists of at least one scaffold material, at least one bioactive agent, peptide #237 and demineralized bone particles. In another embodiment, the composition consists essentially of at least one scaffold material, at least one bioactive agent, peptide #237 and demineralized bone particles.

In one embodiment, the composition comprises at least one scaffold material, at least one bioactive agent, a prominin-1 peptide and demineralized bone particles. In another embodiment, the composition consists of at least one scaffold material, at least one bioactive agent, a prominin-1 peptide and demineralized bone particles. In another embodiment, the composition consists essentially of at least one scaffold material, at least one bioactive agent, a prominin-1 peptide and demineralized bone particles.

In one embodiment, the composition comprises at least one scaffold material, at least one bioactive agent and a prominin-1 peptide. In another embodiment, the composition consists of at least one scaffold material, at least one bioactive agent and a prominin-1 peptide. In another embodiment, the composition consists essentially of at least one scaffold material, at least one bioactive agent and a prominin-1 peptide.

In one embodiment, the composition comprises at least one scaffold material, at least one bioactive agent and demineralized bone particles. In another embodiment, the composition consists of at least one scaffold material, at least one bioactive agent and demineralized bone particles. In another embodiment, the composition consists essentially of at least one scaffold material, at least one bioactive agent and demineralized bone particles.

In one embodiment, the composition comprises at least one scaffold material and demineralized bone particles. In another embodiment, the composition consists of at least one scaffold material and demineralized bone particles. In another embodiment, the composition consists essentially of at least one scaffold material and demineralized bone particles.

In one embodiment, the composition comprises at least one scaffold material, a prominin-1 peptide and demineralized bone particles. In another embodiment, the composition consists of at least one scaffold material, a prominin-1 peptide and demineralized bone particles. In another embodiment, the composition consists essentially of at least one scaffold material, a prominin-1 peptide and demineralized bone particles.

In one embodiment, the composition comprises at least one scaffold material and a prominin-1 peptide. In another embodiment, the composition consists of at least one scaffold material and a prominin-1 peptide. In another embodiment, the composition consists essentially of at least one scaffold material and a prominin-1 peptide.

In one embodiment of the composition, the scaffold material is formulated as a microsphere. In one embodiment of the composition, the at least one scaffold material is formulated as a microsphere. In one embodiment of the composition, the first scaffold material is formulated as a microsphere. It contemplated that the microsphere can be made of any scaffold material known in the art including those described herein. In one embodiment, the microsphere is a PLGA microsphere. In another embodiment, the microsphere is a collagen microsphere.

In one embodiment, the microsphere encapsulates the at least one bioactive agent. For example, a PLGA microsphere encapsulates PDGF within.

In one embodiment, the microsphere is coated externally with the at least one bioactive agent. For example, PDGF encoating a PLGA microsphere.

In one embodiment, the microsphere encapsulates the at least one bioactive agent and is coated externally with the at least one bioactive agent. For example, a PLGA microsphere encapsulates PDGF within, and is coated externally with PDGF.

In one embodiment, the microsphere encapsulates the at least one bioactive agent and is coated externally with a second bioactive agent. For example, a PLGA microsphere encapsulates PDGF within, and is coated externally with VEGF.

In one embodiment, the composition comprises a PLGA microsphere, VEGF, PDGF, peptide #237 and demineralized bone particles, wherein the PLGA microsphere encapsulate PDGF within and is coated with VEGF externally. In another embodiment, the composition consists of a PLGA microsphere, VEGF, PDGF, peptide #237 and demineralized bone particles, wherein the PLGA microsphere encapsulate PDGF within and is coated with VEGF externally. In another embodiment, the composition consists essentially of a PLGA microsphere, VEGF, PDGF, peptide #237 and demineralized bone particles, wherein the PLGA microsphere encapsulate PDGF within and is coated with VEGF externally.

In one embodiment, the composition comprises a PLGA microsphere, PDGF, peptide #237 and demineralized bone particles, wherein the PLGA microsphere encapsulates PDGF within. In another embodiment, the composition consists of a PLGA microsphere, PDGF, peptide #237 and demineralized bone particles, wherein the PLGA microsphere encapsulates PDGF within. In another embodiment, the composition consists essentially of a PLGA microsphere, PDGF, peptide #237 and demineralized bone particles, wherein the PLGA microsphere encapsulates PDGF within.

In one embodiment, the composition comprises a PLGA microsphere, VEGF, peptide #237 and demineralized bone particles, wherein the PLGA microsphere is coated with VEGF externally. In another embodiment, the composition consists of a PLGA microsphere, VEGF, peptide #237 and demineralized bone particles, wherein the PLGA microsphere is coated with VEGF externally. In another embodiment, the composition consists essentially of a PLGA microsphere, VEGF, peptide #237 and demineralized bone particles, wherein the PLGA microsphere is coated with VEGF externally.

In one embodiment, the composition comprises a PLGA microsphere, peptide #237 and demineralized bone particles. In another embodiment, the composition consists of a PLGA microsphere, peptide #237 and demineralized bone particles. It is also contemplated that the peptide #237 can be encapsulated within the PLGA microsphere and/or coats the PLGA microsphere externally. In another embodiment, the composition consists essentially of a PLGA microsphere, peptide #237 and demineralized bone particles. It is also contemplated that the peptide #237 can be encapsulated within the PLGA microsphere and/or coats the PLGA microsphere externally.

In one embodiment, the composition comprises a microsphere, VEGF, PDGF, peptide #237 and demineralized bone particles, wherein the microsphere encapsulate PDGF within and is coated with VEGF externally. In another embodiment, the composition consists of a microsphere, VEGF, PDGF, peptide #237 and demineralized bone particles, wherein the microsphere encapsulate PDGF within and is coated with VEGF externally. In another embodiment, the composition consists essentially of a microsphere, VEGF, PDGF, peptide #237 and demineralized bone particles, wherein the microsphere encapsulate PDGF within and is coated with VEGF externally.

In one embodiment, the composition comprises a microsphere, PDGF, peptide #237 and demineralized bone particles, wherein the microsphere encapsulates PDGF within. In another embodiment, the composition consists of a microsphere, PDGF, peptide #237 and demineralized bone particles, wherein the microsphere encapsulates PDGF within. In another embodiment, the composition consists essentially of a microsphere, PDGF, peptide #237 and demineralized bone particles, wherein the microsphere encapsulates PDGF within.

In one embodiment, the composition comprises a microsphere, VEGF, peptide #237 and demineralized bone particles, wherein the microsphere is coated with VEGF externally. In another embodiment, the composition consists of a microsphere, VEGF, peptide #237 and demineralized bone particles, wherein the microsphere is coated with VEGF externally. In another embodiment, the composition consists essentially of a microsphere, VEGF, peptide #237 and demineralized bone particles, wherein the microsphere is coated with VEGF externally.

In one embodiment, the composition comprises a microsphere, peptide #237 and demineralized bone particles. In another embodiment, the composition consists of a microsphere, peptide #237 and demineralized bone particles. It is also contemplated that the peptide #237 can be encapsulated within the microsphere and/or coats the microsphere externally. In another embodiment, the composition consists essentially of a microsphere, peptide #237 and demineralized bone particles. It is also contemplated that the peptide #237 can be encapsulated within the microsphere and/or coats the microsphere externally.

In one embodiment, the composition comprises a collagen microsphere, VEGF, PDGF, peptide #237 and demineralized bone particles, wherein the collagen microsphere encapsulate PDGF within and is coated with VEGF externally. In another embodiment, the composition consists of a collagen microsphere, VEGF, PDGF, peptide #237 and demineralized bone particles, wherein the collagen microsphere encapsulate PDGF within and is coated with VEGF externally. In another embodiment, the composition consists essentially of a collagen microsphere, VEGF, PDGF, peptide #237 and demineralized bone particles, wherein the collagen microsphere encapsulate PDGF within and is coated with VEGF externally.

In one embodiment, the composition comprises a collagen microsphere, PDGF, peptide #237 and demineralized bone particles, wherein the collagen microsphere encapsulates PDGF within. In another embodiment, the composition consists of a collagen microsphere, PDGF, peptide #237 and demineralized bone particles, wherein the collagen microsphere encapsulates PDGF within. In another embodiment, the composition consists essentially of a collagen microsphere, PDGF, peptide #237 and demineralized bone particles, wherein the collagen microsphere encapsulates PDGF within.

In one embodiment, the composition comprises a collagen microsphere, VEGF, peptide #237 and demineralized bone particles, wherein the collagen micro sphere is coated with VEGF externally. In another embodiment, the composition consists of a collagen microsphere, VEGF, peptide #237 and demineralized bone particles, wherein the collagen microsphere is coated with VEGF externally. In another embodiment, the composition consists essentially of a collagen microsphere, VEGF, peptide #237 and demineralized bone particles, wherein the collagen microsphere is coated with VEGF externally.

In one embodiment, the composition comprises a collagen microsphere, peptide #237 and demineralized bone particles. In another embodiment, the composition consists of a collagen microsphere, peptide #237 and demineralized bone particles. It is also contemplated that the peptide #237 can be encapsulated within the collagen microsphere and/or coats the collagen microsphere externally. In another embodiment, the composition consists essentially of a collagen microsphere, peptide #237 and demineralized bone particles. It is also contemplated that the peptide #237 can be encapsulated within the collagen microsphere and/or coats the collagen microsphere externally.

In one embodiment of the composition, the at least one bioactive agent, the first scaffold material and the at least one other scaffold material are solidified or aggregated together into a fabricated structure having a defined shape, size and volume.

In one embodiment, the composition is solidified or aggregated together by pressure. In one embodiment, the composition is solidified or aggregated together by compression.

In one embodiment of the composition further comprising a solidifying agent for solidifying the at least one bioactive agent, the first scaffold material and the at least one other scaffold material into a fabricated structure having a defined shape, size and volume.

In another embodiment of the composition, the microsphere, the at least one bioactive agent and the at least one scaffold material are solidified or aggregated together into a fabricated structure having a defined shape and size. Various methods of solidifying or aggregating materials in the fabrication process are known in the art, e.g., compression, use of solidifying agent that cross-link the materials to form polymers, e.g., by covalently linkages.

In one embodiment, the one or more scaffold materials of the composition are cross-linked together into a fabricated structure having a defined shape and size. In one embodiment, scaffold materials of the composition comprise a mixture of non-ionically cross-linkable scaffold material with scaffold materials which are ionically cross-linkable, i.e., scaffold material such as the polymers described herein which are not ionically cross-linkable are used in blends with polymers which are ionically cross-linkable.

The cross-linking ions used to crosslink the scaffold materials can be anions or cations depending on whether the scaffold material is anionically or cationically cross-linkable. Appropriate cross-linking ions include but not limited to cations selected from the group consisting of calcium, magnesium, barium, strontium, boron, beryllium, aluminum, iron, copper, cobalt, lead and silver ions. Anions can be selected from but not limited to the group consisting of phosphate, citrate, borate, succinate, maleate, adipate and oxalate ions. More broadly, the anions are derived from polybasic organic or inorganic acids. In some embodiments, the cross-linking cations are calcium, iron, and barium ions. In some embodiments, the cross-linking anion is phosphate. Cross-linking can be carried out by contacting the scaffold materials with a solution containing dissolved ions. One of ordinary skill in the art will be able to select appropriate cross-linking agent for the respective scaffold materials used in the making of a fabricated composition. For example, the gelation of collagen or alginate occurs in the presence of ionic cross-linker or divalent cations such as Ca²⁺, Ba²⁺ and Sr²⁺.

In another embodiment of the composition, further comprising a solidifying agent for solidifying the microsphere, the at least one bioactive agent and the at least one scaffold material into a fabricated structure having a defined shape and size.

In one embodiment, the solidifying agent is a cation or an anion.

In one embodiment, the solidified or aggregated fabricated structure is porous. The porosity of the fabricated structure permits all the materials needed for bone growth and/or regeneration to infiltrate into the structure to lay down new bone materials, e.g., migrating non-differentiated cells, fibrblasts, mesenchymal stem cells, osteoblast cells, osteoid matrix, extracellular matrix etc.

The solidification/aggregation process permits fabrication of the composition into all sorts of sizes and shapes. This then permits the fabricated the composition to fit into the void space presented by a defect in a hard tissue or oral tissue defect. For example, a 10-cm section of a long leg bone femur is shattered to multiple fragments and is beyond salvage. The composition described herein can be fabricated into rod-shape composition of about 9 cm and this rod-shape composition is placed in the 10-cm gap of the femur.

In one embodiment, the composition comprises a PLGA microsphere, VEGF, PDGF, peptide #237, demineralized bone particles and alginate, wherein the PLGA microsphere encapsulate PDGF within and is coated with VEGF externally. In another embodiment, the composition consists of a PLGA microsphere, VEGF, PDGF, peptide #237, demineralized bone particles and alginate, wherein the PLGA microsphere encapsulate PDGF within and is coated with VEGF externally.

In one embodiment, the composition comprises a PLGA microsphere, PDGF, peptide #237, demineralized bone particles and alginate, wherein the PLGA microsphere encapsulate PDGF within. In another embodiment, the composition consist of a PLGA microsphere, PDGF, peptide #237, demineralized bone particles and alginate, wherein the PLGA microsphere encapsulate PDGF within.

In one embodiment, the composition comprises a PLGA microsphere, peptide #237 demineralized bone particles and alginate. In another embodiment, the composition consists of a PLGA microsphere, peptide #237 demineralized bone particles and alginate. It is also contemplated that the peptide #237 can be encapsulated within the PLGA microsphere and/or coats the PLGA microsphere externally.

In certain embodiments, the composition described herein contains additional components, such as insoluble collagen, other extracellular matrix proteins (ECM), such as proteoglycans and glycosaminoglycans, fibronectin, laminin, entectin, decorin, lysyl oxidase, cros slinking precursors (reducible and non-reducible), elastin, elastin cros slink precursors, cell components such as, cell membrane proteins, mitochondrial proteins, nuclear proteins, cytosomal proteins, and cell surface receptors, growth factors, such as, PDGF, TGF, EGF, and VEGF, and hydroxyproline.

In certain embodiments, the composition described herein contains inactive materials such as buffering agents and pH adjusting agents such as potassium bicarbonate, potassium carbonate, potassium hydroxide, sodium acetate, sodium borate, sodium bicarbonate, sodium carbonate, sodium hydroxide or sodium phosphate; degradation/release modifiers; drug release adjusting agents; emulsifiers; preservatives such as benzalkonium chloride, chlorobutanol, phenylmercuric acetate and phenylmercuric nitrate, sodium bisulfite, sodium bisulfate, sodium thiosulfate, thimerosal, methylparaben, polyvinyl alcohol and phenylethyl alcohol; solubility adjusting agents; stabilizers; and/or cohesion modifiers. Typically, any such inactive materials will be present within the range of 0-75 wt %, and more typically within the range of 0-30 wt %. In certain embodiments, the composition described herein comprises sterile preservative free material.

In one embodiment, the composition is sterile for in vivo use.

In one embodiment, the composition described herein is used for bone growth and/or bone regeneration in any hard tissue defects or oral tissue defects. In another embodiment, the composition described herein is used for tissue growth, tissue repair and/or tissue regeneration in any soft tissue defects or oral tissue defects.

### Bioactive Agents

In some embodiments, the compositions for promoting bone growth and/or bone regeneration are incorporated with bioactive agents. These bioactive agents stimulate cell growth, migration of differentiated and non-differentiated cells, and the differentiation of non-differentiated cells (e.g., progenitor and stem cells) towards and at the repair, regeneration or new growth site. Progenitor cells that are typically involved include endothelial progenitor cells (EPCs) and mesenchymal progenitor cells (MPCs).

A great number of growth factors and differentiation factors that are known in the art to stimulated cell growth and differentiation of the progenitor and stem cells. Suitable growth factors and cytokines include any cytokines or growth factors capable of stimulating, maintaining, and/or mobilizing cells. They include but not limited to stem cell factor (SCF), granulocyte-colony stimulating factor (G-CSF), granulocyte-macrophage stimulating factor (GM-CSF), stromal cell-derived factor-1, steel factor, vascular endothelial growth factor (VEGF), transforming growth factor-beta (TGFβ), platelet derived growth factor (PDGF), angiopoeitins (Ang), epidermal growth factor (EGF), bone morphogenic protein (BMP), fibroblast growth factor (FGF), hepatocye growth factor, insulin-like growth factor (IGF-1), interleukin (IL)-3, IL-1α, IL-1β, IL-6, IL-7, IL-8, IL-11, and IL-13, colony-stimulating factors, thrombopoietin, erythropoietin, fit3-ligand, and tumor necrosis factor α (TNF-α). Other examples are described in Dijke et al., “Growth Factors for Wound Healing”, Bio/Technology, 7:793-798 (1989); Mulder G D, Haberer P A, Jeter K F, eds. Clinicians' Pocket Guide to Chronic Wound Repair. 4th ed. Springhouse, Pa.: Springhouse Corporation; 1998:85; Ziegler T. R., Pierce, G. F., and Herndon, D. N., 1997, International Symposium on Growth Factors and Wound Healing: Basic Science & Potential Clinical Applications (Boston, 1995, Serono Symposia USA), Publisher: Springer Verlag.

Examples of growth factors include EGF, bFGF, HNF, NGF, PDGF, IGF-1 and TGF-β. These growth factors can be mixed with the scaffold materials comprising the compositions.

In one embodiment, the bioactive agents of the compositions described herein have pro-angiogenic activities, e.g., VEGF, PDGF, and prominin-1 polypeptide and peptides thereof that have pro-angiogenic activities, i.e., promote neovascularization and angiogenesis.

In one embodiment, the bioactive agents of the compositions described herein promote or stimulate bone growth. By way of example only, the bioactive agent is bone morphogenic proteins (BMPs); they display pro-osteogenic properties.

In certain embodiments, several bioactive agents are incorporated into the compositions. Any bioactive agent displaying an ability to enhance tissue repair, regeneration, or healing is encompassed. In certain embodiments, the bioactive agent is encapsulated within the scaffold material for purpose of sustained release at the site of bone repair or growth. In other embodiments, the bioactive agent encoats the scaffold material for purpose of an initial high dose release at the site of bone repair or growth.

In some embodiments, active forms of particular bioactive agents are for use in the composition. For example, proteins that are naturally synthesized as pre-proteins, prepro-proteins, or other pre-modified forms that are not fully active are preferably administered in processed or modified forms that are active. Alternatively, if inactive forms of bioactive agents are applied, then additional agents may be co-administered or included in the composition to activate of the bioactive agents. For example, a protease can be co-administered with PDGF-C in order to cleave the CUB domain and activate the protein. “Activation” is understood as a processing of a protein or peptide so that it is able to bind to and/or activate a receptor.

In some embodiments where the bioactive agents are polypeptide and peptides, the polypeptide and peptide sequences are modified. In some embodiments, the modification comprises conservative substitutions, insertions, and/or deletions, wherein the modified polypeptide and peptides still retain the biological activity of the native bioactive agent. Modification methods are known in the art. Standard modification methods include but are not limited to site-directed mutagenesis. Similarly, use of peptidomimetic compounds or compounds in which one or more amino acid residues are replaced by a non-naturally-occurring amino acid or an amino acid analog that retains the required aspects of the biological activity of the native bioactive agent is contemplated.

In some embodiments, variant forms of bioactive agent polypeptides are used in the compositions. In some embodiments, variant forms of bioactive agent polypeptides result from alternative splicing and naturally-occurring allelic variation of the nucleic acid sequence encoding a bioactive agent. Allelic variants are well known in the art, and represent alternative forms or a nucleic acid sequence that comprise substitution, deletion or addition of one or more nucleotides, but which do not result in any substantial functional alteration of the encoded polypeptide.

In other embodiments, variant forms of bioactive agent peptides are prepared by targeting non-essential regions of that polypeptide for modification. In the case of the VEGF and PDGF, the non-essential regions are expected to fall outside the strongly-conserved regions of the VEGF/PDGF family of growth factors. In particular, the growth factors of the PDGF/VEGF family, including VEGF-B and the PDGFs, are dimeric, and at least VEGF-A, VEGF-B, VEGF-C, VEGF-D, PDGF-A and PDGF-B show complete conservation of eight cysteine residues in the N-terminal domains, i.e. the PDGF/VEGF-like domains (Olofsson, et al., Proc. Nat'l. Acad. Sci. USA, 1996 93:2576-2581; Joukov, et al., EMBO J., 1996 15:290-298, both incorporated herein by reference in their entirety). These cysteines are thought to be involved in intra- and inter-molecular disulfide bonding. In addition there are further strongly, but not completely, conserved cysteine residues in the C-terminal domains. Loops 1, 2 and 3 of each subunit, which are formed by intra-molecular disulfide bonding, are involved in binding to the receptors for the PDGF/VEGF family of growth factors (Andersson, et al., Growth Factors, 1995 12:159-64). These references are incorporated herein by reference in their entirety.

These conserved cysteine residues are preferably preserved in any proposed variant form, although there can be exceptions, because receptor-binding VEGF-B analogs are known in which one or more of the cysteines are not conserved. Similarly, the active sites present in loops 1, 2 and 3 also should be preserved. Other regions of the molecule can be expected to be of lesser importance for biological function, and therefore offer suitable targets for modification. Modified polypeptides can readily be tested for their ability to show the biological activity of VEGF-B or a PDGF.

Preferably, where amino acid substitution is used, the substitution is conservative, i.e., an amino acid is replaced by one of similar size and with similar charge properties. As used herein, the term “conservative substitution” denotes the replacement of an amino acid residue by another, biologically similar residue. Examples of conservative substitutions include the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic acid for aspartic acid, or glutamine for asparagine, and the like. Neutral hydrophilic amino acids that can be substituted for one another include asparagine, glutamine, serine and threonine. In one embodiment, only one of the amino acids is substituted with a conservative substitution. In another embodiment, two substitutions can be made. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. Conservative amino acid substitutions do not change the overall structure of the peptide nor the type of amino acid side chains available for forming van der Waals bonds with a binding partner. Conservative amino acid substitution can be achieved during chemical synthesis of the peptide by adding the desired substitute amino acid at the appropriate sequence in the synthesis process. Alternatively, molecular biology methods can be used. The term “conservative substitution” also includes the use of a substituted amino acid in place of an unsubstituted parent amino acid. Alternatively, conservative amino acids can be grouped as described in Lehninger, (Biochemistry, Second Edition; Worth Publishers, Inc. NY:NY, pp. 71-77 (1975)) as set out in the following: Non-polar (hydrophobic) A. Aliphatic: A, L, I, V, P, B. Aromatic: F, W, C. Sulfur-containing: M, D. Borderline: G. Uncharged-polar A. Hydroxyl: S, T, Y, B. Amides: N, Q, C. Sulfhydryl: C, D. Borderline: G. Positively Charged (Basic): K, R, H. Negatively Charged (Acidic): D, E.

In other embodiments, a bioactive agent polypeptide can be modified, for instance, by glycosylation, amidation, carboxylation, or phosphorylation, or by the creation of acid addition salts, amides, esters, in particular C-terminal esters, and N-acyl derivatives. The proteins also can be modified to create peptide derivatives by forming covalent or noncovalent complexes with other moieties. Covalently bound complexes can be prepared by linking the chemical moieties to functional groups on the side chains of amino acids comprising the peptides, or at the N- or C-terminus.

### Platelet-Derived Growth Factor (PDGF)

In one embodiment, the at least one bioactive agent of the composition is a PDGF.

At least four distinct platelet-derived growth factor (PDGF) family members have been identified, PDGF-A (GENBANK™ Accession Nos: GI:77695917, SEQ ID NO: 01; GI:15208656, SEQ ID NO: 02; GI:80476673, SEQ ID NO: 03), PDGF-B (GENBANK™ Accession Nos: GI: 4505681, SEQ ID NO: 04; GI: 15451786, SEQ ID NO: 05; GI: 2297379, SEQ ID NO: 06; GI: 2297379, SEQ ID NO: 07), PDGF-C (GENBANK™ Accession Nos: GI: 9994187, SEQ ID NO: 08; GI:218091978, SEQ ID NO: 09), and PDGF-D (GENBANK™ Accession Nos: GI: 13376808, SEQ ID NO: 10; GI:15451921, SEQ ID NO: 11). In some embodiments, natural PDGF peptides are used. In some embodiments, human PDGF peptides are used. The term PDGF comprises, but is not limited to PDGF-A, PDGF-B, PDGF-C, and PDGF-D, or a fragment or analog thereof having the ability to bind PDGF-receptors. Active analogs should exhibit at least 85% sequence identity, preferably at least 90% sequence identity, particularly preferable at least 95% sequence identity, and especially preferable at least 98% sequence identity to the natural PDGF polypeptides. In some embodiments “PDGF” refers to all isoforms, variants, homologs, derivatives and peptides of the PDGF family.

PDGF plays an important role in embryonic development, cell proliferation, cell migration and angiogenesis, which is very important for periodontal tissue development, maintenance and regeneration (Meraw et al., J. Periodontol 2000; 71 (1):8-13). PDGF has been used to promote the regeneration of several periodontal tissues, including gingiva, alveolar bone and cementum (Park et al., J. Periodontol 1995; 66 (6):462-77) and has a stimulatory effect on human periodontal ligament (HPDL) cells, which is important for periodontal ligament formation (Fujita et al., Cell Biol. Int. 2004; 28 (4):281-6). PDGF-CC is known to enhance post-ischemic revascularization in the heart and limb, apparently by mobilizing endothelial progenitor cells, inducing differentiation of bone marrow cells into endothelial cells, stimulating migration of endothelial cells, and upregulating VEGF expression. Moreover, PDGF-CC induces the differentiation of bone marrow cells into smooth muscle cells (SMC) and stimulates SMC growth and migration during vessel sprouting (US Patent Application 2004/0248796).

PDGF-A and PDGF-B were characterized first in the literature and have thus been the subject of a greater body of research and development. Homo- and heterodimers have been formed with these polypeptides, and variants have been described with altered amino acid sequences yet the same or similar receptor binding properties. Exemplary PDGF-A and -B polypeptides for use in the compositions and methods described herein have been described in U.S. Pat. Nos. 5,605,816 (PDGF-A and A/B heterodimers); 4,889,919 (PDGF-A homodimers); 5,759,815 (recombinant production of PDGF-A or -B in prokaryotes and formation of various dimers); 5,889,149 (PDGF-AB isoforms); 4,845,075 and 5,428,010 and 5,516,896 (PDGF-BB homodimers); 5,272,064 and 5,512,545 (PDGF-B analogues); 5,905,142 (protease-resistant PDGF-B analogues); and 5,128,321 and 5,498,600 and 5,474,982 (PDGF-A/B mosaics). These documents are all incorporated by reference in their entirety. In addition to the foregoing patent documents, there is substantial scientific literature describing and characterizing PDGF-A and -B proteins.

In a preferred embodiment of the compositions, the PDGF polypeptide is a recombinant human PDGF-BB (rhPDGF-BB).

In other embodiments, the PDGF polypeptide comprises a PDGF-C or PDGF-D polypeptide. PDGF-C polypeptides and polynucleotides were characterized by Eriksson et al. in International Patent Publication No. WO 00/18212, U.S. Patent Application Publication No. 2002/0164687, and U.S. patent Ser. No. 10/303,997. PDGF-D polynucleotides and polypeptides were characterized by Eriksson, et al. in International Patent Publication No. WO 00/27879 and U.S. Patent Application Publication No. 2002/0164710. These documents are all incorporated by reference in their entirety. PDGF-C and -D bind to PDGF receptors alpha and beta, respectively.

In certain embodiments, a variant of PDGF-C comprises the PDGF/VEGF homology domain (PVHD) of PDGF-C and retains receptor binding and activation functions, as described in US Patent Application 2004/0248796 which is incorporated by reference in its entirety. In certain embodiments, a variant of PDGF-D comprises the PDGF/VEGF homology domain (PVHD) of PDGF-D and retains receptor binding and activation functions, as described in US Patent Application 2004/0248796 which is incorporated by reference in its entirety.

In addition to naturally occurring PDGF polypeptides, variant forms that still bind to and/or the respective PDGF receptors (including receptor homodimers and heterodimers) also contemplated. Variants with at least 90% amino acid sequence identity to a naturally occurring human PDGF-A -B, -C, or -D polypeptide are preferred. Still more preferred is at least 92%, 94%, 95%, 96%, 97%, 98%, or 99% sequence identity. Thus, in another variation, the PDGF polypeptide comprises a portion of the amino acid sequence set forth in GENBANK™ Accession Nos: GI: 197333758, SEQ ID NO: 12; GI: 197333759, SEQ ID NO: 13; GI: 208879461, SEQ ID NO: 14; GI: 208879462, SEQ ID NO: 15; GI: 307691204, SEQ ID NO: 16; GI: 78190489, SEQ ID NO: 17; GI: 78190476, SEQ ID NO: 18 that is effective to bind PDGFR-α and/or PDGFR-β.

PDGF polypeptides are available commercially from R&D Systems, Inc. (Minneapolis, Minn.) Human PDGF-BB Cat. #220-BB-010, Human PDGF-AA Cat. #221-AA-010, Human PDGF Cat. #120-HD-001, Human PDGF-CC Cat. #1687-CC-025, Human PDGF-DD Cat. #1159-SB-025, Human PDGF-AB Cat. #222-AB-010. Alternatively, PDGF can be prepared according to the methods of US Patent Application 2004/0248796. Sequences of the various PDGFs can be obtained form GENBANK™.

### Vascular Endothelial Growth Factor (VEGF)

In one embodiment, the at least one bioactive agent of the composition is VEGF.

The VEGF subfamily is composed of members that share a VEGF homology domain (VHD) characterized by the sequence: C-X(22-24)-P-[PSR]-C-V-X(3)-R-C-[GSTA]-G-C-C-X(6)-C-X(32-41)-C (SEQ ID NO: 19), wherein X may be any amino acid. The VHD domain, determined through analysis of the VEGF subfamily members, comprises the PDGF motif but is more specific. The VEGF subfamily of growth factors and receptors regulate the development and growth of the vascular endothelial system. VEGF family members include VEGF-A, VEGF-B (GENBANK™ Accession No: GI:4507887; SEQ ID NO: 20), VEGF-C (GENBANK™ Accession No: GI: 4885653; SEQ ID NO: 21), VEGF-D (GENBANK™ Accession No: GI: 4758378; SEQ ID NO: 22) and PLGF (GENBANK™ Accession No: GI:20149543; SEQ ID NO: 23) (Li, X. and U. Eriksson, Int. J. Biochem. Cell. Biol., 2001 33:421-6, incorporated herein by reference in its entirety). As used herein “VEGF” refers to all isoforms, variants, derivatives and peptides of the VEGF family, proteins having the same amino acid sequence as a naturally-occurring VEGF family protein, and also fragments, analogs, or variants that have sequence variation (e.g., alternatively spliced forms), yet retain VEGF biological activity. Examples of VEGF-B analogs are described in WO 98/28621 and in Olofsson, et al., Proc. Nat'l. Acad. Sci. USA, 95:11709-11714 (1998), both incorporated herein by reference. Examples of PDGF analogs are described in U.S. Pat. Nos. 5,512,545, and 5,474,982; U.S. Patent Application Nos.: 20020164687 and 20020164710. These references are incorporated herein by reference in their entirety

VEGF-A (or VEGF) was originally purified from several sources on the basis of its mitogenic activity toward endothelial cells, and also by its ability to induce microvascular permeability, hence it is also called vascular permeability factor (VPF). VEGF-A has subsequently been shown to induce a number of biological processes including the mobilization of intracellular calcium, the induction of plasminogen activator and plasminogen activator inhibitor-1 synthesis, promotion of monocyte migration in vitro, induction of antiapoptotic protein expression in human endothelial cells, induction of fenestrations in endothelial cells, promotion of cell adhesion molecule expression in endothelial cells and induction of nitric oxide mediated vasodilation and hypotension (Ferrara, J. Mol. Med. 1999 77: 527-543; Neufeld, et al., FASEB. J 1999 13:9-22; Zachary, Intl. J. Biochem. Cell. Bio. 1998 30:1169-74; incorporated herein by reference in their entirety).

VEGF-A is a secreted, disulfide-linked homodimeric glycoprotein composed of 23 kD subunits. Five human VEGF-A isoforms of 121 (GENBANK™ Acc. No. GI: 284172469; SEQ ID NO: 24), 145 (GENBANK™ Acc. No. GI: 324120930; SEQ ID NO: 25), 165 (GENBANK™ Acc. No. GI: 284172465; SEQ ID NO: 26), 189 (GENBANK™ Acc. No. GI: 284172461; SEQ ID NO: 27) or 206 (GENBANK™ Acc. No. GI: 284172459; SEQ ID NO: 28) amino acids in length (VEGF₁₂₁-VEGF₂₀₆), encoded by distinct mRNA splice variants, have been described, all of which are capable of stimulating mitogenesis in endothelial cells. However, each isoform differs in biological activity, receptor specificity, and affinity for cell surface- and extracellular matrix-associated heparan-sulfate proteoglycans, which behave as low affinity receptors for VEGF-A. VEGF₁₂₁ does not bind to either heparin or heparan-sulfate; VEGF₁₄₅ and VEGF₁₆₅ (GENBANK™ Acc. No. M32977) are both capable of binding to heparin; and VEGF₁₈₉ and VEGF206 show the strongest affinity for heparin and heparan-sulfates. VEGF₁₂₁, VEGF₁₄₅, and VEGF₁₆₅ are secreted in a soluble form, although most of VEGF₁₆₅ is confined to cell surface and extracellular matrix proteoglycans, whereas VEGF₁₈₉ and VEGF₂₀₆ remain associated with extracellular matrix. Both VEGF₁₈₉ and VEGF₂₀₆ can be released by treatment with heparin or heparinase, indicating that these isoforms are bound to extracellular matrix via proteoglycans. Cell-bound VEGF₁₈₉ can also be cleaved by proteases such as plasmin, resulting in release of an active soluble VEGF₁₁₀. Most tissues that express VEGF are observed to express several VEGF isoforms simultaneously, although VEGF₁₂₁ and VEGF₁₆₅ are the predominant forms, whereas VEGF₂₀₆ is rarely detected (Ferrara, J. Mol. Med. 1999 77:527-543). VEGF₁₄₅ differs in that it is primarily expressed in cells derived from reproductive organs (Neufeld et al., FASEB. J 1999 13:9-22).

In a preferred embodiment of the composition, the VEGF is recombinant human VEGF₁₆₅ (rhVEGF₁₆₅). Alternatively, the VEGF is VEGF₁₆₄ (NCBI Ref Seq: NP_{—}033531; SEQ ID NO: 29), a recombinant mouse homolog of human VEGF₁₆₅.

In one embodiment of the composition, the VEGF is a VEGF-B or isoform thereof.

The term “VEGF-B” as used herein encompasses those polypeptides identified as VEGF-B in U.S. Pat. No. 6,331,301, U.S. Patent Publication 2003/0008824 which are incorporated herein by reference in their entirety. VEGF-B comprises, but is not limited to, both the VEGF-B₁₆₇ and/or VEGF-B₁₈₆ isoforms or a fragment or analog thereof having the ability to bind VEGFR-1. Active analogs should exhibit at least 85% sequence identity, preferably at least 90% sequence identity, particularly preferably at least 95% sequence identity, and especially preferably at least 98% sequence identity to the natural VEGF-B polypeptides, as determined by BLAST analysis. The active substance typically will include the amino acid sequence Pro-Xaa-Cys-Val-Xaa-Xaa-Xaa-Arg-Cys-Xaa-Gly-Cys-Cys (SEQ ID NO: 30) (where Xaa may be any amino acid) that is characteristic of VEGF-B.

In another embodiment of the composition, the VEGF is a VEGF-C or isoform thereof. VEGF-C comprises a VHD that is approximately 30% identical at the amino acid level to VEGF-A. VEGF-C is originally expressed as a larger precursor protein, prepro-VEGF-C, having extensive amino- and carboxy-terminal peptide sequences flanking the VHD, with the C-terminal peptide containing tandemly repeated cysteine residues in a motif typical of Balbiani ring 3 protein. Prepro-VEGF-C undergoes extensive proteolytic maturation involving the successive cleavage of a signal peptide, the C-terminal pro-peptide, and the N-terminal pro-peptide. Secreted VEGF-C protein consists of a non-covalently-linked homodimer, in which each monomer contains the VHD. The intermediate forms of VEGF-C produced by partial proteolytic processing show increasing affinity for the VEGFR-3 receptor, and the mature protein is also able to bind to the VEGFR-2 receptor. (Joukov, et al., EMBO J, 1997 16:3898-3911; International Patent Publication No. WO 98/33917; these references are incorporated herein by reference in their entirety).

In another embodiment of the composition, the VEGF is a VEGF-C or isoform thereof. VEGF-D is structurally and functionally most closely related to VEGF-C (See International Patent Publ. No. WO 98/07832, incorporated herein by reference in its entirety). Like VEGF-C, VEGF-D is initially expressed as a prepro-peptide that undergoes N-terminal and C-terminal proteolytic processing, and forms non-covalently linked dimers. VEGF-D stimulates mitogenic responses in endothelial cells in vitro. During embryogenesis, VEGF-D is expressed in a complex temporal and spatial pattern, and its expression persists in the heart, lung, and skeletal muscles in adults. Isolation of a biologically active fragment of VEGF-D designated VEGF-DANAC, is described in International Patent Publication No. WO 98/07832, incorporated herein by reference. VEGF-DANAC consists of amino acid residues 93 to 201 of VEGF-D linked to the affinity tag peptide FLAG®.

In another embodiment of the composition, the VEGF is VEGF-E, NZ2 VEGF or VEGF-like proteins from virus, D1701 and NZ10. These additional members of the VEGF subfamily have been identified in poxviruses, which infect humans, sheep and goats. The orf virus-encoded VEGF-E and NZ2 VEGF are potent mitogens and permeability enhancing factors. Both show approximately 25% amino acid identity to mammalian VEGF-A, and are expressed as disulfide-liked homodimers. Infection by these viruses is characterized by pustular dermatitis which may involve endothelial cell proliferation and vascular permeability induced by these viral VEGF proteins. (Ferrara, J. Mol. Med. 1999 77:527-543; Stacker and Achen, Growth Factors 1999 17:1-11). VEGF-like proteins have also been identified from two additional strains of the orf virus, D1701 (described in Meyer, et al., EMBO. J 1999 18:363-374, incorporated herein by reference in its entirety) and NZ10 (described in International Patent Application PCT/US99/25869, incorporated herein by reference in its entirety). These viral VEGF-like proteins have been shown to bind VEGFR-2 present on host endothelium, and this binding is important for development of infection and viral induction of angiogenesis (Meyer, et al., EMBO. J 1999 18:363-74; International Patent Application PCT/US99/25869; incorporated herein by reference in their entirety).

In one embodiment, the VEGF is glycosylated. Exemplary glycosylated VEGF-B forms are described in published U.S. Patent Publication 2002/0068694 and U.S. Pat. Nos. 5,607,918, 5,840,693, and 5,928,939, all incorporated by reference herein in their entirety. In some embodiments, the VEGF polypeptide has an amino acid sequence at least 85% or 90% identical to a natural human VEGF sequence. Still more preferred are those polypeptides that are 91, 92, 93, 94, 95, 96, 97, 98, 99, or 100% identical at the amino acid sequence level with a naturally-occurring human VEGF sequence.

In one embodiment, VEGF sequences modified with conservative substitutions, insertions, and/or deletions. Standard methods can readily be used to generate such polypeptides including site-directed mutagenesis of VEGF polynucleotides, or specific enzymatic cleavage and ligation. Similarly, use of peptidomimetic compounds or compounds in which one or more amino acid residues are replaced by a non-naturally-occurring amino acid or an amino acid analog that retains the required aspects of the biological activity of VEGF is contemplated.

Methods of producing VEGF are known in the art. For example, the isolation and characteristics of VEGF-B, including nucleotide and amino acid sequences for both human and murine VEGF-B, are described in detail in PCT/US96/02957, and U.S. Pat. Nos. 5,840,693 and 5,607,918, US Patent Application 2004/0248796 and in Olofsson, et al., Proc. Natl. Acad. Sci. USA, 93:2576-2581 (1996). These disclosures are incorporated herein by reference in their entirety.

VEGF polypeptides are available commercially from R&D Systems, Inc. (Minneapolis, Minn.) Recombinant Human VEGF₁₆₅ Cat. #293-VE, Human VEGF Cat. #298-VS-005, Human VEGF₁₁₁ Cat #5336-VE-010, Human VEGF₁₂₁ Cat #4644-VS-010, Human VEGF₁₆₂ Cat #2347-VE-025. Alternatively, VEGF may be prepared according to the methods of US Patent Application 2004/0248796.

### Prominin-1 Peptides

In one embodiment, the at least one bioactive agent of the composition is a prominin 1 peptide. In particular, the prominin 1 peptides have pro-angiogenic activity.

As described in PCT/US2009/051971, incorporated herein by reference in its entirety, short peptides of Prominin-1 (prom-1) bind VEGF, an endogenous pro-angiogenesis factor that is important for normal growth and development. Some of the peptides promoted VEGF binding to other cell types, promoted proliferation of endothelial and melanoma cells in vitro, and enhanced angiogenesis and cell migration in the presence of VEGF. These peptides with pro-angiogenic properties are useful in promoting angiogenesis such as in wound healing and tissue repair. The peptides described in Application PCT/US2009/051971 were identified based on their ability to bind VEGF, such as VEGF₁₆₅, VEGF₁₂₁, and VEGF₁₄₅. The peptides potentiate the activity of VEGF in processes including angiogenesis, cell migration, vasodilation, and cell proliferation.

The human prominin-1 (aka AC133, CD133, MSTP061, PROML1, RP41, prominin 1, hProminin, prominin (mouse)-like 1, hematopoietic stem cell antigen) is a penta span transmembrane glycoprotein (5-TMD) expressed in stem cells, primarily on the apical membrane of epithelial cells, and is a marker of hematopoietic stem cells. It belongs to a molecular family of 5-transmembrane domain (TMD) proteins, pfam prominin. This “family” includes members from several different species including human, mouse, rat, fly, zebrafish and nematode worms. The 5-TMD structure includes an extracellular N-terminus, two short intracellular loops, two large extracellular loops and an intracellular C-terminus. Prominin-1 was initially shown to be expressed on primitive hematopoietic stem and progenitor cells and on retinoblastoma cells. However, prominin-1 has since been shown to be expressed on hemangioblasts, and neural stem cells as well as on developing epithelia. The prominin-1 positive fractions of human bone marrow, cord blood and peripheral blood efficiently engraft in xenotransplantation models, and contain the majority of the granulocyte/macrophage precursors, NOD/SCID repopulating cells and CD34+ dendritic cell precursors. Phenotypically, prominin-1 positive cells in blood and marrow are CD34 bright, with CD34 dim/CD71 bright cells being negative for prominin-1 expression. Prominin-1 is also found in extracellular membrane particles in body fluids. No natural ligand has yet been demonstrated for prominin-1, and its specific function in hematopoietic tissue is unknown (Corbeil, D., et. al, Blood. 1998, 91:2625-6; Miraglia S, et. al., Blood. 1997, 90:5013-21; Weigmann A, et. al, Proc Natl. Acad. Sci. USA. 1997, 94:12425-30). The exact function of prominin-1 is unknown although in humans, defects in PROM1, the gene coding for prominin-1, cause retinal degeneration.

In one embodiment, the prominin 1 peptide with pro-angiogenic activity is derived from the full length human prominin-1. In other embodiments, the prominin 1 peptide with pro-angiogenic activity is from homologous protein members of the prominin protein family (pfam05478:Prominin). Homologous protein members of the prominin protein family include known and identified proteins as well as predicted proteins from genomic studies. All members of the prominin family are predicted to contain five membrane spanning domains, with an N-terminal domain exposed to the extracellular space followed by four, alternating small cytoplasmic and large extracellular loops and a cytoplasmic C-terminal domain. These proteins are homologs of the human prominin-1. Examples of prominin-1 homologs are: Danio rerio (zebrafish) prominin-like 2 protein, swissprot Q90WI3, Genbank Accession No. GI:82177379; Rattus norvegicus (Norway rat) testosterone-regulated prominin-related protein, swissprot Q8R4B6, Genbank Accession No. GI:81866961; Homo sapiens (human) Prominin-like protein 2, swissprot Q8N271, Genbank Accession No. GI:74728673; Mus musculus (house mouse) Prominin-like protein 1, swissprot 054990, Genbank Accession No. GI:13124464; Caenorhabditis elegans hypothetical protein F08B12.1, swissprot Q19188, Genbank Accession No. GI:74963586; and Drosophila melanogaster (fruit fly) Prominin-like protein, swissprot P82295, Genbank Accession No. GI:13124468.

In one embodiment, the prominin-1 peptide with pro-angiogenic activity comprises at least 6 consecutive amino acid residues. The 6 amino acid residues are consecutive, reflecting what is encoded and expressed in the intact full length prominin-1 polypeptide. In another embodiment, the prominin-1 peptide with pro-angiogenic activity comprises no more than 50 amino acids of prominin-1. In yet another embodiment, the isolated peptide with pro-angiogenic activity has at least 7, 8, 9, 10, 11, 12, 13, 14, 15, 16, 17, 18, 19, 20, 21, 22, 23, 24, 25, 26, 27, 28, 29, or 30 consecutive amino acid residues of prominin-1. In another embodiment, prominin-1 peptide has no more than 30 consecutive amino acid residues. In yet another embodiment, prominin-1 peptide has no more than 29, 28, 27, 26, 25, 24, 23, 22, 21, 20, 19, 18, 17, 16, 15, 14, 13, 12, 11, 10, 9, 8, or 7 consecutive amino acid residues of prominin-1. In another embodiment, the peptide has 50 or fewer consecutive amino acids and includes peptides with 49, 48, 47, 46, 45, 44, 43, 42, 41, 40, 39, 38, 37, 36, 35, 34, 33, 32, 31, and 30 consecutive amino acids of prominin-1.

In one aspect, prominin-1 peptide with pro-angiogenic activity consists essentially of or consists of a peptide selected from the group consisting of: LCGNSFSGGQPS (SEQ ID NO:31); PNIIPVLDEIKS (SEQ ID NO: 32); LCGVCGYDRHAT (SEQ ID NO: 33); ITNNTSSVIIEE (SEQ ID NO: 34); DRVQRQTTTVVA (SEQ ID NO: 35); and CSFAYDLEAKANSLPPGNLRN (SEQ ID NO: 36) or a conservative substitution variant thereof.

In a preferred embodiment, the prominin-1 peptide is prom-1 #237 having the sequence DRVQRQTTTVVA (SEQ ID NO: 35).

In one embodiment, a fusion polypeptide comprising a prominin-1 peptide or a conservative amino acid substitution variant thereof is contemplated herein. The fusion polypeptide is formed by the fusion of a peptide described herein with another heterologous protein or a portion thereof. The heterologous protein is any protein that is not a member of the prominin family. The fusion gives rise to a prominin-1 chimeric polypeptide. Such fusion prominin-1 peptides can serve to enhance the serum half life of the prominin-1 peptide in vivo. Examples include fusion with albumin, transferrin, transthyretin, and Fc of IgG (See G. M. Subramanian, 2007, Nature Biotechnology 25, 1411-141). Other fusions can facilitate protein expression, solubility during expression, and purification, e.g. thioredoxin, glutathione S-transferase, avidin and six histidine tags. In another embodiment, a peptide or a conservative amino acid substitution variant thereof described herein with pro-angiogenic activity can be fused with other pro-angiogenic factors, e.g. VEGF, FGF and IGF to enhance angiogenic potency.

Prominin-1 polypeptides are available commercially from Bioclone Inc. (San Diego, Va.) Cat. No. PV-0198, Abnova (Taipei, Taiwan) Cat. No. P1650, or ProteinTech Group (Chicago, Ill.) Cat. No. 19945-1-AR Alternatively, prominin-1 can be prepared according to the methods of PCT/US2010/051971.

### Fabricated Scaffolds

In one embodiment, the composition described herein comprises at least one scaffold material. In one embodiment, the scaffold material is solidified together to form a fabricated structure. The fabricated structure is a scaffold. In one embodiment, the fabricated structure has a define shape, size and volume. In one embodiment, the fabricated structure is fabricated to fit the void space formed by a tissue defect, e.g., missing length or portion of a long bone. The scaffold, when implanted into a host, induces and directs the growth of new tissue. The goal is for the cells to attach to the scaffold, then replicate, differentiate, and organize into normal healthy tissue as the scaffold degrades. In some embodiments, only part of the scaffold degrades while other parts become a component of the new tissue.

In certain embodiments of the compositions, two or more scaffold materials are used.

In some embodiments, the scaffold materials are ideally pharmaceutically acceptable biodegradable and/or any bioabsorbable materials that are preferably FDA approved or GRAS materials. These materials can be polymeric or non-polymeric, as well as synthetic or naturally occurring, or a combination thereof.

In some embodiments, the scaffold materials are absorbable or non-absorbable materials. In some embodiments, the scaffold is bioresorbable. “Bioresorbable” refers to the ability of a scaffold to be resorbed or remodeled in vivo. The resorption process involves degradation and elimination of the original material through the action of body fluids, enzymes, or cells. The resorbed material may be used by the treated individual in the formation of new tissue, or it may be otherwise re-utilized by the treated individual, or it may be excreted. A scaffold, in some embodiments, can be resorbed within one year of in vivo administration. In other embodiments, a scaffold can be resorbed within 1, 3, 6, or 9 months of in vivo administration. Bioresorbability is dependent on: (1) the nature of the scaffold material (i.e., its chemical make up, physical structure, and size); (2) the location within the body in which the scaffold is placed; (3) the amount of scaffold material that is used; (4) the metabolic state of the individual being treated (diabetic/non-diabetic, osteoporotic, smoker, age, steroid use, etc.); (5) the extent and/or type of injury or condition treated; and (6) the use of other materials in addition to the scaffold such as other bone anabolic, catabolic, and anti-catabolic factors.

Suitable absorbable materials include glycolide, lactide, trimethylene carbonate, dioxanone, caprolactone, alklene oxides, ortho esters, polymers and copolymers thereof, collagen, hyaluronic acids, alginates, and combinations thereof. Suitable non-absorbable materials include, polypropylene, polyethylene, polyamide, polyalkylene therephalate (such as polyethylene therephalate polybutylene therephalate), polyvinylidene fluoride, polytetrafluoroethylene and blends and copolymers thereof.

In one embodiment, the scaffold material is a biocompatible scaffold material. A scaffold fabricated with biocompatible materials provides an improved substrate for cell attachment. In one embodiment, the biocompatible scaffold material used is bioabsorbable.

Suitable biocompatible materials that can be used to envelope the scaffold include absorbable or non-absorbable materials or a combination thereof. Suitable absorbable materials include those stated hereinabove. Suitable non-absorbable materials include those non-absorbable materials stated hereinabove. In some embodiments, the scaffold is embedded or encased in a bioabsorbable material.

In one embodiment, the scaffold material is synthetic in origin. In other embodiments, the scaffold materials are natural materials: in particular different derivatives of the extracellular matrix have been studied to evaluate their ability to support cell growth. Protein based materials, such as collagen or fibrin, and polysaccharidic materials, like chitosan or glycosaminoglycans (GAGs), have all proved suitable in terms of cell compatibility, but some issues with potential immunogenicity still remains. Among GAGs hyaluronic acid, possibly in combination with cross linking agents (e.g. glutaraldehyde, water soluble carbodiimide, etc.), is one of the possible choices as scaffold material. Functionalized groups of scaffolds can be useful in the delivery of small molecules (drugs) to specific tissues.

In certain embodiments, the scaffold material is a bioabsorbable, bioerodible, and/or a biodegradable biopolymer that provides immediate release, sustained release or controlled release of the bioactive agents. Examples of suitable sustained release biopolymers include, but are not limited to, poly(alpha-hydroxy acids), poly(lactide-co-glycolide) (PLGA), polylactide (PLA), polyglycolide (PG), polyethylene glycol (PEG), PEG 200, PEG 300, PEG 400, PEG 500, PEG 550, PEG 600, PEG 700, PEG 800, PEG 900, PEG 1000, PEG 1450, PEG 3350, PEG 4500, PEG 8000, conjugates of poly(alpha-hydroxy acids), polyorthoesters, polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D,L-lactide, or L-lactide, -caprolactone, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG (poly(d,l-lactide-co-glycolide), PLA-PLGA, poloxamer 407, PEG-PLGA-PEG triblock copolymers, SAIB (sucrose acetate isobutyrate) hydroxypropyl cellulose, hydroxypropyl methylcellulose, hydroxyethyl methylcellulose, carboxymethylcellulose or salts thereof, Carbopol, poly(hydroxyethylmethacrylate), poly(methoxyethylmethacrylate), poly(methoxyethoxy-ethylmethacrylate), polymethylmethacrylate (PMMA), methylmethacrylate (MMA), gelatin, polyvinyl alcohols, propylene glycol, or combinations thereof. In other embodiments, the scaffold material is not a bioabsorbable, bioerodible, and/or a biodegradable biopolymer.

A variety of scaffolds comprising various scaffold materials are described in U.S. Pat. Nos. 6,103,255, 6,224,893, 6,228,117, 6,328,990, 6,376,742, 6,432,435, 6,514,515, 6,525,145, 6,541,023, 6,562,374, 6,656,489, 6,689,166, 6,696,575, 6,737,072, 6,902,932 and WO/2005/110050, these are hereby incorporated by reference it their entirety.

In one embodiment, the scaffold material is selected from the non-limiting group consisting of glycosaminoglycan, silk, fibrin, MATRIGEL™, poly-ethyleneglycol (PEG), polyhydroxy ethyl methacrylate, polyvinyl alcohol, polyacrylamide, poly(N-vinyl pyrolidone), poly glycolic acid (PGA), poly lactic-co-glycolic acid (PLGA), poly e-carpolactone (PCL), polyethylene oxide, poly propylene fumarate (PPF), poly acrylic acid (PAA), hydrolysed polyacrylonitrile, polymethacrylic acid, polyethylene amine, alginic acid, pectinic acid, carboxy methyl cellulose, hyaluronic acid, heparin, heparin sulfate, chitosan, carboxymethyl chitosan, chitin, pullulan, gellan, xanthan, collagen, gelatin, carboxymethyl starch, carboxymethyl dextran, chondroitin sulfate, cationic guar, cationic starch as well as salts and esters thereof.

In one embodiment, a scaffold is a temporary device, and the scaffold material is mostly or entirely resorbed by the body of a subject having a fabricated scaffold implanted therein. In such embodiments, tissue eventually takes up at least a portion of physical space occupied by the scaffold. For example, in repairing a bone defect, a porous material is often used to allow for bone ingrowth into the graft material. Porous bone grafts act as a scaffold or trellis that allows regenerating bone to heal across a defect that it normally could not. In certain embodiments, these grafts can be provided in small particles within the composition that become a permanent component of the new tissue growth.

In one embodiment, the composition is a porous fabricated scaffold, with pores having diameters ranging from about 1 um to about 1 mm. In certain embodiments, a porous fabricated scaffold comprises macropores having diameters ranging from about 100 um to about 1 mm. In certain embodiments, a scaffold comprises mesopores having diameters ranging from about 10 um to about 100 um. In certain embodiments, a porous fabricated scaffold comprises micropores having diameters less than about 10 um. In certain embodiments, the porous fabricated scaffold can comprise macropores, mesopores, micropores, or any combination thereof. A porous fabricated scaffold, in one embodiment, includes a scaffold with a porosity of about or greater than any of 25, 30, 40, 50, 60, 70, 75, 80, 85, 90, or 95%. In certain embodiments, the porous structure of the scaffold allows for infiltration of cells (such as osteoblasts) into pores of the scaffold. In some embodiments, a scaffold comprises a porous structure having multidirectional and/or interconnected pores. In other embodiments, a scaffold comprises a porous structure having pores that are not interconnected. In some embodiments, a scaffold comprises a porous structure having a mixture of interconnected pores and pores that are not interconnected. In some embodiments, a scaffold is porous and able to absorb water in an amount ranging from about 1 to about 15 times the mass of the scaffold.

In certain embodiments, the scaffold material is a bone fragment or a bone-derived material/fragment. In one embodiment, the bone fragment or bone-derived material/fragment is not biodegradable and/or any bioabsorbable. The bone fragment or bone-derived material/fragment becomes a component of the new tissue formed at the tissue defect site (i.e. site where repair or regeneration occurred).

In certain embodiments, the bone fragment or bone-derived material/fragment includes bone allograft, isograft, autograft, and xenograft. Bone allografts include bone or bone cells from a donor that can be transplanted into a genetically non-identical member of the same species. Transplanted bone or bone cells from a genetically identical donor, i.e., an identical twin, is termed an isograft. When a cell or tissue is transplanted from one site to another in the same individual, it is termed an autograft. In contrast, a transplant from another species is called a xenograft. In some cases, bone from a human donor is transplanted into another human. Exemplary human bone allografts are pieces of bone skeleton isolated post mortem from human donors. In certain embodiments, the bone fragment or bone-derived material/fragment is mineralized or partially or completely demineralized using standard methods. In certain embodiments, the bone fragment or bone-derived material/fragment is non-demineralized. In certain embodiments, the bone fragment or bone-derived material/fragment is deorganified using standard methods. In certain embodiments, the bone fragment or bone-derived material/fragment is non-demineralized and deorganified. In certain embodiments, the bone fragment or bone-derived material/fragment contains a combination of (i) mineralized bone and (ii) partially or completely demineralized bone. In certain embodiments, the bone fragment or bone-derived material/fragment is partially or completely deproteinized using standard methods, such as a deproteinized bovine or human bone block. Exemplary bone fragments or bone-derived material/fragments include a demineralized freeze-dried bone graft (DFDBA), a freeze-dried bone graft (FDBA), a fresh frozen bone allograft, a particulate demineralized bone matrix (DBM), or a bone block (see, for example, U.S. Ser. No. 60/890,763; U.S. Pub. No. 2007/0207185; U.S. Pub. No. 2007/0129807; which are each incorporated herein by reference in their entireties). In certain embodiments, the bone fragment or bone-derived material/fragment is an autologous cortical, cancellous, or cortico-cancellous bone block. In certain embodiments, the bone fragment or bone-derived material/fragment is a deorganified xenogeneic material, e.g., BioOss (Geistlich Biomaterials, Inc.). In some embodiments, the bone fragment or bone-derived material/fragment is a commercially prepared bone graft for use in humans. In some embodiments, the bone fragment or bone-derived material/fragment has been treated so that it is suitable for use in humans. Exemplary treatment steps to make a bone fragment or bone-derived material/fragment suitable for use in humans include, but are not limited to, bioburden control, bioburden assessment, minimized contamination, rigorous cleaning, disinfection and rinsing, milling, freeze drying, aliquotting, packaging, terminal sterilization, mineralization, demineralization, freeze drying, aseptic preparation, bone block or granulate formation, or any combination of two or more of the foregoing. In some embodiments, the bone fragment or bone-derived material/fragment undergoes Allowash XG®. (bioburden control, bioburden assessment, minimized contamination, rigorous cleaning, disinfection, and rinsing). In various embodiments, the bone fragment or bone-derived material/fragment is milled or not milled, freeze-dried or not freeze-dried, sterilized or just aseptically prepared. In some embodiments, the bone fragment or bone-derived material/fragment is treated with one or more chemicals (such as hydrogen peroxide, detergent surfactants such as nonoxynyl-9, or isopropyl alcolol) or antibiotics (such as polymyxin or bacitracin). In some embodiments, the bone fragment or bone-derived material/fragment is exposed either to gamma radiation or to ethylene oxide for sterilization. Not all bone fragments or bone-derived material/fragments are terminally sterilized. In some embodiments, bone fragment or bone-derived material/fragment is treated to remove viruses and/or bacteria.

Exemplary bone fragments or bone-derived material/fragments are derived from one or more of the following types of bone: humerus, ulna, radius, femur, tibia, fibula, patella, ankle bones, wrist bones, carpals, metacarpals, phalanges, tarsals, metatarsals, ribs, sternum, vertebrae, scapula, clavicle, pelvis, sacrum, and craniofacial bones. Exemplary donors for bone scaffold include a primate (e.g., a human, monkey, gorilla, ape, lemur, etc.), a bovine, an equine, a porcine, an ovine, a canine, and a feline.

In some embodiments, the bone fragment or bone-derived material/fragment is comprised of particles, blends, meshes, or blocks. Any appropriate overall size of bone fragment or bone-derived material/fragment can be used, such as an overall size useful to treat a bone defect or injury of a particular size. In some embodiments, the bone fragment or bone-derived material/fragment comprises of particulates of any appropriate size, such as between about 50 and about 750 um, about 50 and about 500 um, about 125 and about 500 um, about 250 and about 710 um, or about 125 and about 1000 um. In some embodiments, the bone scaffold consists of a bone block with an average diameter between about 50 um and about 100 mm or about 0.1 mm and about 100 mm. In some embodiments, the particulates are less than about 100 um (such as between about 50 and about 90 um) or greater than 355 um (such as between about 360 and about 1000 um) since bone scaffold with a particle size between about 100 and about 355 um may be less flowable than desired for some applications. Flowability refers to the ability to pass the material through a cannula or small gauge tube as a homogeneous mixture, that is, without the separation of the liquid from the particulate. In some embodiments, a broad size range (such as between about 250 and about 710 um) is used to maximize the yield from the bone scaffold. For example, ground cortical bone is processed on standard grinding equipment that produces particulate in a range of sizes. The broader the size range that is allowable, the greater the yield.

In one embodiment of the composition, the bone fragment or bone-derived material/fragment is composed of mineralized or demineralized freeze dried bone allograft.

In some embodiments, resorbable polymers, ceramics, and composites, which have been shown to be effective substitutes for bone derived grafts, can be used. These scaffolds are characterized by a high percent porosity to allow for bone and/or cartilage in-growth. A variety of pore forming techniques used to create three-dimensional porous scaffolds are known and include those disclosed in U.S. Pat. Nos. 7,458,991, 7,087,200, 6,887,488, 6,993,406, 7,022,522, 7,005,135, 6,379,962, 7,875,342 and 7,241,486. US Patent Publications 2009/0292359, 2010/0272826, 2005/0113934, 2010/0249931, 2008/0060725, 2009/0292359, 2010/0226956, 2005/0158535, 2007/0178159, 2010/0137990, 2010/0292146, 2010/0272693, 2010/0016989, 2007/0213424, 2008/0281431, 2003/0082808, 2003/0003127, 2010/0330144, 2007/0134346 and 2004/0191292 which are incorporated herein by reference in their entirety.

In certain embodiments, the scaffold material is collagen or ALLODERM®. Compositions comprising collagen or ALLODERM® are useful for enhancing the repair/regeneration of a soft tissue defect. The collagen is synthetic or naturally derived. Natural sources of collagen are obtained from animal or human sources. For instance, it is derived from rat, pig, cow, or human tissue or tissue from any other species. Tendons, ligaments, muscle, fascia, skin, cartilage, tail, or any source of collagenous tissue are useful. Alternatively the collagen is obtained from autologous or allogenic cells. For instance, the collagen is derived from a subject's fibroblasts which have been cultured. The collagen is then be used in that subject or other subjects. The fabricated scaffold is prepared, by varying the collagen content and other components to provide the desired viscosity of the finished composition. In some embodiments, the fabricated scaffold has a collagen viscosity of 1,000 to 200,000 centipoise. Methods for preparing collagen scaffolds are known in the art and include the techniques described in US Patent Publication 2009/0254104 which is incorporated herein by reference in its entirety.

In some embodiments, the scaffold material forms a fabricated scaffold that take the form of meshes, other filamentous structures, non-woven, sponges, woven or non-woven materials, knit or non-knit materials, felts, salt eluted porous materials, molded porous materials, 3D-printing generated scaffolds, foams, perforated sheets, grids, parallel fibers with other fibers crossing at various degrees, and combinations thereof. The fabricated scaffold can be in a variety of different sizes, shapes and configurations including sheets, cylinders, tubes, spheres or beads. There are several factors that can be taken into consideration in determining the size, shape and configuration of the fabricated scaffold. For example, both the size and shape may allow for ease in positioning the fabricated scaffold at the target tissue site that is selected as the implantation site. In addition, the shape and size of the fabricated scaffold should be selected so as to minimize or prevent the fabricated scaffold from moving after implantation or injection. In various embodiments, the fabricated scaffold can be shaped like a sphere, a cylinder such as a rod or pellet, fiber, a flat surface such as a disc, film, or sheet, or the like. Flexibility may be a consideration so as to facilitate placement of the fabricated scaffold.

In one embodiment, the scaffold material forms a fabricated scaffold that takes the form of a microsphere. In one embodiment, the scaffold material is PLGA and the scaffold material forms a PLGA microsphere. In one embodiment, the PLGA microsphere comprises bioactive agents. Scaffold materials form making microspheres and methods of making the microspheres are well known in the art, e.g., U.S. Pat. Nos. 4,331,654, 5,271,961, and 6,919,685. These are incorporated herein by reference in their entirety.

In a preferred embodiment, microspheres of PLGA are prepared according to the methods described in US Patent Publication 2004/0026811; Mooney et al., Biomaterials 17, 1417-1422 (1996); Richardson and Mooney, In Methods of tissue engineering. (eds Atala, A. & Lanza, R.) 653-662 (Academic Press, San Diego, Calif.; 2001); and Cohen et al., Pharmacol. Res. 8, 713-720 (1991), all of which are incorporated herein by reference in their entirety. Briefly, microspheres of PLGA are made by standard double-emulsion with PDGF and prominin-1 peptide being added during this process, thus encapsulating the PDGF and prominin-1 peptide inside the microspheres. After being lyophilization, the microspheres are mixed with VEGF and mineralized FDBA fragments for the matrix preparation. Using a gas foaming process (Harris, L. D., et al., J Biomed Mater Res 42:396, 1998, incorporated herein by reference in its entirety), the PLGA microsphere are mixed with porogen particles, VEGF and mineralized FDBA fragments and the mixture is compressed into a solid mixture of a discontinuous polymer with an interspersed porogen, VEGF and mineralized FDBA fragments. The resulting pellet is exposed to high pressure CO₂ gas, and after the pressure is allowed to equilibrate over a period of time the pressure is rapidly released causing a thermodynamic instability in the polymer component of the mixture. The instability causes the polymer to foam, and the originally discontinuous polymer particles fuse together to form a continuous polymer scaffold around interspersed porogen particles, which are then leached out in a solvent. In each of these processes the degree of interconnection between pores in the resulting scaffold is determined by the interconnection of porogen particles during the solvent evaporation or polymer foaming steps, respectively. Because the porogen is dispersed within the polymer before fusion, the degree of interconnection between porogen particles is not actively controlled and the interconnectivity of pores in the final scaffold is uncontrolled. In this particular embodiment, following implantation in the subject, the scaffold releases VEGF in a burst mode and PDGF and prominin-1 peptide in a more sustained/prolonged timeframe. Also the fabricated structure allows for cell migration.

In one embodiment, the scaffold material forms a hydrogel. In one embodiment, the scaffold material is PLGA and the scaffold material forms a hydrogel. In one embodiment, the PLGA hydrogel comprises bioactive agents. Scaffold materials form making hydrogel are well known in the art. Suitable hydrogels include but are not limited to natural hydrogels, such as for example, gelatin, collagen, silk, elastin, fibrin and polysaccharide-derived polymers like agarose, and chitosan, glucomannan gel, hyaluronic acid, polysaccharides, such as cross-linked carboxyl-containing polysaccharides, or a combination thereof. Synthetic hydrogels include, but are not limited to those formed from polyvinyl alcohol, acrylamides such as polyacrylic acid and poly(acrylonitrile-acrylic acid), polyurethanes, polyethylene glycol (e.g., PEG 3350, PEG 4500, PEG 8000), silicone, polyolefins such as polyisobutylene and polyisoprene, copolymers of silicone and polyurethane, neoprene, nitrile, vulcanized rubber, poly(N-vinyl-2-pyrrolidone), acrylates such as poly(2-hydroxy ethyl methacrylate) and copolymers of acrylates with N-vinyl pyrolidone, N-vinyl lactams, polyacrylonitrile or combinations thereof. The hydrogel materials may further be cross-linked to provide further strength as needed. Examples of different types of polyurethanes include thermoplastic or thermoset polyurethanes, aliphatic or aromatic polyurethanes, polyetherurethane, polycarbonate-urethane or silicone polyether-urethane, or a combination thereof. Methods of making the hydrogel are well known in the art, e.g., U.S. Pat. Nos. 5,212,044, 5,226,902, 5,346,935, 6,878,384, 6,899,896, and 7,799,352. These are incorporated herein by reference in their entirety.

### Tissue Healing, Repair and Regeneration

In one embodiment, provided herein is a method of promoting bone growth and/or bone regeneration in a hard tissue or an oral tissue, the method comprising contacting the tissue with a composition described herein. In another embodiment, the method comprises implanting the composition into the tissue. In another embodiment, the method comprises shaping the composition to fit into the tissue and then implanting the composition into the tissue.

In another embodiment, provided herein is a method of treating or repairing a hard tissue defect or an oral tissue defect in a subject, the method comprising contacting the defect with a composition described herein. In another embodiment, the method comprises implanting the composition into the tissue. In another embodiment, the method comprises shaping the composition to fit into the tissue, taking the shape of the defect, and then implanting the composition into the tissue.

In another embodiment, provided herein is a method of treating or repairing a hard tissue defect or an oral tissue defect in a subject, the method comprising selecting a subject having the defect and contacting the defect in the subject with a composition described herein. In another embodiment, the method comprises implanting the composition into the tissue. In another embodiment, the method comprises shaping the composition to fit into the tissue, taking the shape of the defect, and then implanting the composition into the tissue.

In some embodiments, the composition is placed, implanted or transplanted into, on, or proximal to a target tissue (e.g., tissue with a defect needing repair, tissue with missing portion thereof) wherein that target tissue resides either internal or external (skin surfaces) to the body. The composition is introduced into or onto a bodily tissue using a variety of known methods and tools that are known in the art e.g., tweezers or graspers, hypodermic needle, and endoscopic manipulator. The composition can be assembled in place, for example by sequentially injecting or inserting the composition.

In certain embodiments, the composition is designed to cause an initial burst dose of bioactive agent and/or scaffold material within the first 24 hours after implantation. “Initial burst” or “burst effect” or “bolus dose” refers to the release of bioactive agent and/or scaffold material from the composition during the first 24 hours after the composition comes in contact with an aqueous fluid. In certain embodiments, the composition is designed to avoid this initial burst effect.

In certain embodiments, the composition contains one or more different release layer(s) that releases a bolus dose of a bioactive agent and/or scaffold material and one or more sustained release layer(s) that releases an effective amount of a bioactive agent and/or scaffold material over a period of, for example, 1 to 8 weeks. In certain embodiments, the one or more immediate release layer(s) comprise PLGA, which degrades faster and than the one or more sustained release layer(s), which comprises PLA, which degrades at a slower rate than the PLGA.

In various embodiments, the composition releases 5%, 10%, 15%, 20%, 25%, 30%, 40%, 50%, 60%, 70%, 80%, 90%, 95%, or 99% of a bioactive agent or scaffold material over a period of 1 to 8 weeks or 2 to 6 weeks after the composition is administered to the target tissue site to enhance tissue repair, healing, or regeneration. The composition has a “release rate profile” that refers to the percentage of a bioactive agent or scaffold material that is released over fixed units of time, e.g., μg/hr, mg/hr, μg/day, mg/day, 10% per day for one week, ten days, etc. As persons of ordinary skill know a release rate profile may be but need not be linear.

In one exemplary embodiment, the composition comprises about 0.01 to 1 ug/uL each of VEGF, PDGF, and prom-1 #237 and releases an effective amount of the bioactive agents and scaffold over a period of at least 1 week to 6 weeks. In certain embodiments, the composition comprises PLGA microspheres.

In another exemplary embodiment, the composition comprises (i) PLGA microspheres having a diameter between about 5-50 μm in diameter (ii) about 0.1 μg/μL VEGF, (ii) about 0.15 μg/μL PDGF, (iii) about 100 μg/μL prominin-1, and releases an effective amount of the bioactive agents over a period of at least 1 week to 6 weeks. In one embodiment, the PLGA microspheres encapsulate PDGF and the PDGF in released from it. In one embodiment, VEGF externally coats the PLGA microspheres and is released from it in a bolus dose.

In certain embodiments, the composition is used in the context of tissue engineering, tissue regeneration, tissue repair, regenerative medicine and wound healing in a subject. Tissue engineering aims at developing functional cell, tissue, and organ substitutes to repair, replace or enhance biological function that has been lost due to congenital abnormalities, injury, disease, or aging, or repair tissues. The tissue that is engineered is used to repair or replace portions of or whole tissues (i.e., bone, cartilage, blood vessels, bladder, etc. . . . ). Often, the tissues involved require certain mechanical and structural properties for proper function.

Tissue regeneration aims to restore and repair tissue function via the interplay of living cells, an extra-cellular matrix and cell communicators. Tissue regeneration is an approach in modern medicine that delivers living tissue or cells and stimulates the body's own natural healing process by activating the body's inherent ability to repair and regenerate. Innovative tissue regeneration therapies are now available that aim to heal or reconstruct diseased tissue and support the regeneration of diseased or injured organs. Doctors use tissue regeneration to speed up healing and to help injuries that will not heal or repair on their own. Tissue regeneration can help heal broken bones, severe burns, chronic wounds, heart damage, nerve damage, and many other diseases.

In certain embodiments, the composition is used to enhance tissue healing, repair and/or regeneration. In certain embodiments it may be used as a graft or implant to replace a portion of a subject's bone or bone system, cartilage, or teeth. For example, the composition can be used to repair a fracture or other bony defect in a subject's bone. Bone defects include any area of bone tissue that is inadequate for cosmetic or physiological purposes. Bone defects may be caused by birth defect, trauma, disease, decay, or surgery. Bone implants are often applied at a bone defect site, e.g., one resulting from injury, defect brought about during the course of surgery, infection, malignancy, inflammation, or developmental malformation.

In certain embodiments, bone grafting is the replacement or augmentation of the bone around the teeth. Bone grafting is performed to reverse the bone loss/destruction caused by periodontal disease, trauma, or ill fitting removable dentures. It is also used to augment bone to permit implant placement, such as augmenting bone in the sinus area for implant placement, or augmenting bone to enhance the fit and comfort of removable prostheses, or to enhance esthetics of a missing tooth site in the smile zone. When one loses a tooth, as in an extraction, the surrounding bone collapses. To preserve this bone for future implant placement or for esthetics, a bone graft is used.

The composition described herein can be used in a variety of orthopedic, neurosurgical, plastic, reconstructive, dental, and oral and maxillofacial surgical procedures such as the repair of simple and compound fractures and non-unions, external, and internal fixations, joint reconstructions such as arthrodesis, general arthroplasty, deficit filling, disectomy, laminectomy, anterior cerival and thoracic operations, spinal fusions, etc. Specific examples include, but are not limited to, repair of simple and compound fractures and non-unions, external and internal fixations, joint reconstructions such as arthrodesis, general arthroplasty, cup arthroplasty of the hip, femoral and humeral head replacement, femoral head surface replacement and total joint replacement, repairs of the vertebral column including spinal fusion and internal fixation, tumor surgery, e.g. deficit filling, discectomy, laminectomy, excision of spinal cord tumors, anterial cervical and thoracic operations, repair of spinal injuries, scoliosis, lordosis and kyphosis treatments, intermaxillary fixation of fractures, mentoplasty, temporomandibular joint replacement, alveolar ridge augmentation and reconstruction, inlay bone grafts, implant placement and revision, and sinus lifts.

In certain embodiments the bone degeneration or injury may be associated with a cancer. Examples of a cancer are an osteosarcoma, multiple myeloma or a breast or prostate cancer metastasizing to the bone. In another aspect the bone degeneration or injury may be associated with osteoporosis, osteogenesis imperfecta, or severe cranial injury.

In certain embodiments, the composition described herein serves as osteoconductive scaffold for supporting new bone formation.

In certain embodiments, the composition described herein is used to treat a bone injury, disease, or disorder without the necessity for prosthetic reconstruction, or the requirement for donor bone tissue. Thus, the methods and compositions described herein have a great utility in simplifying the treatment of bone injury. Furthermore, the methods can dramatically accelerate the healing of less severe fractures, allowing recipients to regain mobility after a shorter duration.

In certain embodiments, the composition is utilized to enhance oral tissue repair or regeneration. Examples of oral tissue defects include, but are not limited to dentoalveolar defects, connective tissue defects, or collagen membrane defects. In certain embodiments, the composition is utilized during connective tissue grafts or sinus lift procedures.

In a further embodiment, the composition is utilized as an implant or graft during a guided bone regeneration procedure. During guided bone regeneration, barrier membranes are utilized to direct the growth of both hard tissue and prevent unwanted soft tissue or scar tissue growth. Guided bone regeneration methods are described in more depth in U.S. Pat. No. 6,616,698 and US Patent Publications 2001/0014831 and 2010/0028409.

In a further embodiment, the composition is utilized during a sinus lift procedure. Sinus lift is one of the most commonly performed bone grafting procedures for patients with bone loss in the upper jaw (maxilla). The goal of the procedure is to grow bone in the floor of the maxillary sinus above the alveolar ridge—the bony ridge of the gumline that anchors the teeth in the upper jaw. A sinus lift is performed when a dental implant is necessary in the upper molar area of the mouth and the alveolar crestal bone is too thin and does not provide sufficient mass or volume to properly anchor an implant. A graft is performed to enhance the amount of bone available as a base for the needed dental implant. A number of sinus lift techniques are known in the art and include those discussed in U.S. Pat. Nos. 5,397,235, 7,662,188, 7,125,253, 5,547,378, 5,711,315, and US Patent Publications 2008/0319466, 2010/0291511, 2010/0203473 2010/0324561, 2005/0021142, 2010/0255446 and 2010/0081112 which are hereby incorporated by reference in their entirety.

The composition described herein can have a shape that allows the implant to match the bone or tissue defect that the implant is used to replace. The composition may have a circular, oval, elongated disk, ring, square, rectangular, or irregular cross-sectional shape. In other embodiments, the composition may be U-shaped, C-shaped, an elongated ring with a gap, a disk with an orifice, or an elongated disk with an orifice. In other embodiments, the composition could be molded, shaped, or injected into the site of implantation.

The composition for tissue repair can be designed to fill and partially wrap around a defect site. In one embodiment, the composition is shaped or sized smaller than the tissue defect. In such embodiment, a single composition or several compositions in the defect site are used. The surgeon can match the contour of the composition with the contour of the defect and insert the composition into the void. In an embodiment where several compositions are used to provide strength of a defect, the tissue repair compositions can be oriented to maximize strength of the repair.

In a further embodiment, the composition described herein is used as a coating for an implant or graft. The implant or graft can be directed towards the repair of a hard tissue or soft tissue defect and can be composed of any of the graft or implant materials discussed herein. In a further embodiment the implant or graft is a dental implant or graft and is a bone, tooth, connective tissue, or soft tissue material or a synthetic substitute for any of those tissues as described herein.

In certain embodiments, the application of the composition described herein can induce angiogenesis and tissue growth within the transplanted tissue and/or graft and recipient tissue. In one embodiment, the composition can enhance the ability of the graft to take hold in the host tissue. The application of the compositions described herein occurs during the surgery before the subject is sutured up.

In certain embodiments, the composition described herein further comprises cells, e.g., EPCs, MPCs and osteoblasts.

In certain embodiments, the composition described herein is used in conjunction with an engineered tissue. In this embodiment, the composition induces angiogenesis and tissue growth within the transplanted engineered tissue and recipient tissue. In one embodiment, the tissue engineering for tissue repair and tissue repair can be performed in an autologous individual from whom the donor cells were derived, or in HLA type matched individual, which is HLA typed matched with the donor of the cells. In an autologous system, the cells should match exactly the recipient subject from whom the cells are originally derived. In another embodiment, the cells are not used in an autologous system but are HLA typed match to the recipient. For example, the HLA type matched for HLA-A, B, C, and D.

In certain embodiments, the subject is a multicellular organism. The subject can be a plant, an animal or even a developing embryo. In one embodiment, the subject is a mammal. In one embodiment, the subject is a primate mammal. In another embodiment, the subject is a non-primate mammal. In one embodiment, the subject is a domesticated farm animal. In one embodiment, the subject is a cultivated plant, shrub or tree. Examples of a subject are a human, a cat, a dog, a cow, or a horse.

In another embodiment, provided herein are kits for the treatment of tissue defects and kits for promoting bone growth or regeneration. The kit comprises a composition described herein. The composite is preferably sterilely packaged. In certain embodiments, the entire kit is sterilely packaged for use in a sterile environment such as an operating room. Various amounts of the composition can be packaged in a kit. For larger implantation sites, kits with greater amounts of the composition are used. The amount of the composition packaged in a kit may depend on the procedure being performed on the subject. In certain embodiments, multiple individually packaged amounts of the composition are included in one kit. That way only the necessary number of packages need be opened for a procedure. In one embodiment, the kit further comprises a solvent or pharmaceutically acceptable excipient for combining with the composition. In a further embodiment, the kit comprises instructions for using the compositions described herein.

Some embodiments of the present invention can be defined as any of the following numbered paragraphs:
1. A composition for promoting bone growth and/or bone regeneration comprising at least one bioactive agent, a first scaffold material, and at least one other scaffold material.
2. The composition of paragraph 1, wherein the at least one bioactive agent is an agent that promotes angiogenesis.
3. The composition of paragraph 1, wherein the at least one bioactive agent is an agent that promotes bone growth.
4. The composition of paragraph 2, wherein the at least one bioactive agent is selected from the group consisting of vascular endothelial growth factor (VEGF), platelet-derived growth factor (PDGF), fibroblast growth factor (FGF), and prominin-1 peptide (peptide #237).
5. The composition of paragraph 3, wherein the at least one bioactive agent is selected from the group consisting of bone morphogenetic protein 2 (BMP2), BMP4, and BMP7.
6. The composition of paragraph 1, wherein the first scaffold material is a bone fragment or a bone-derived fragment.
7. The composition of paragraph 6, wherein the bone fragment is a demineralized bone particle.
8. The composition of paragraph 1, wherein the first scaffold material is selected from the group consisting of demineralized bone particles, mineralized bone particles, demineralized freeze-dried bone particles, mineralized freeze-dried bone particles, ceramic particles, cancellous chips, and collagen.
9. The composition of paragraph 1, wherein the at least one other scaffold material is selected from the group consisting of glycosaminoglycan, silk, fibrin, MATRIGEL™, poly-ethyleneglycol (PEG), polyhydroxy ethyl methacrylate, polyvinyl alcohol, polyacrylamide, poly(N-vinyl pyrolidone), poly glycolic acid (PGA), poly lactic-co-glycolic acid (PLGA), poly e-carpolactone (PCL), polyethylene oxide, poly propylene fumarate (PPF), poly acrylic acid (PAA), hydrolysed polyacrylonitrile, polymethacrylic acid, polyethylene amine, alginic acid, pectinic acid, carboxy methyl cellulose, hyaluronic acid, heparin, heparin sulfate, chitosan, carboxymethyl chitosan, chitin, pullulan, gellan, xanthan, collagen, gelatin, carboxymethyl starch, carboxymethyl dextran, chondroitin sulfate, cationic guar, and cationic starch as well as salts and esters thereof.
10. The composition of paragraph 1, wherein the at least one other scaffold material is poly (lactic-co-glycolic acid) (PLGA).
11. The composition of paragraph 1, wherein the at least one other scaffold material forms a microsphere.
12. The composition of paragraph 11, wherein the microsphere encapsulates the at least one bioactive agent.
13. The composition of paragraph 11, wherein the microsphere is coated externally with the at least one bioactive agent.
14. The composition of paragraph 11, wherein the microsphere encapsulates the at least one bioactive agent and is coated externally with the at least one bioactive agent.
15. The composition of paragraph 11, wherein the microsphere encapsulates the at least one bioactive agent and is coated externally with a second bioactive agent.
16. The composition of paragraph 1 comprising PLGA, VEGF, PDGF, peptide #237 (prom-1 #237), and demineralized bone particles.
17. The composition of paragraph 1 comprising PLGA, PDGF, peptide #237 (prom-1 #237), and demineralized bone particles.
18. The composition of paragraph 1 comprising PLGA, VEGF, peptide #237 (prom-1 #237), and demineralized bone particles.
19. The composition of paragraph 1 comprising PLGA, peptide #237 (prom-1 #237), and demineralized bone particles.
20. The composition of paragraph 1, wherein the at least one bioactive agent, the first scaffold material, and the at least one other scaffold material are solidified together into a fabricated structure having a defined shape and size.
21. The composition of paragraph 1, further comprising a solidifying agent for solidifying the at least one bioactive agent, the first scaffold material, and the at least one other scaffold material into a fabricated structure having a defined shape and size.
22. The composition of paragraph 21, wherein the solidifying agent is alginate.
23. The composition of paragraph 20, wherein the solidified fabricated structure is porous.
24. A composition for promoting bone growth and/or bone regeneration comprising a microsphere, at least one bioactive agent and at least one scaffold material.
25. The composition of paragraph 24, wherein the microsphere is a poly(lactic-co-glycolic acid) (PLGA) microsphere.
26. The composition of paragraph 24, wherein the microsphere encapsulates the at least one bioactive agent.
27. The composition of paragraph 24, wherein the microsphere is coated externally with the at least one bioactive agent.
28. The composition of paragraph 24, wherein the microsphere encapsulates the at least one bioactive agent and is coated externally with the at least one bioactive agent.
29. The composition of paragraph 24, wherein the microsphere encapsulates the at least one bioactive agent and is coated externally with a second bioactive agent.
30. The composition of paragraph 24, wherein the at least one bioactive agent is an agent that promotes angiogenesis.
31. The composition of paragraph 24, wherein the at least one bioactive agent is an agent that promotes bone growth.
32. The composition of paragraph 30, wherein the at least one bioactive agent is selected from the group consisting of vascular endothelial growth factor (VEGF), platelet-derived growth factor (PDGF), fibroblast growth factor (FGF), and peptide #237 ((prom-1 #237).
33. The composition of paragraph 31, wherein the at least one bioactive agent is selected from the group consisting of bone morphogenetic protein 2 (BMP2), BMP4, and BMP7.
34. The composition of paragraph 24, wherein the at least one scaffold material is a bone fragment.
35. The composition of paragraph 24, wherein the bone fragment is demineralized bone particles.
36. The composition of paragraph 24, wherein the at least one scaffold material is selected from the group consisting of demineralized bone particles, mineralized bone particles, demineralized freeze-dried bone particles, mineralized freeze-dried bone particles, ceramic particles, cancellous chips and collagen.
37. The composition of paragraph 24, comprising a PLGA microsphere, VEGF, PDGF, peptide #237 (prom-1 #237) and demineralized bone particles, wherein the PLGA microsphere encapsulates PDGF within and is coated with VEGF externally.
38. The composition of paragraph 24, comprising a PLGA microsphere, PDGF, peptide #237 (prom-1 #237) and demineralized bone particles, wherein the PLGA microsphere encapsulates PDGF within.
39. The composition of paragraph 24, comprising a PLGA microsphere, peptide #237 (prom-1 #237) and demineralized bone particles.
40. The composition of paragraph 24, wherein the microsphere, the at least one bioactive agent, and the at least one scaffold material are solidified together into a fabricated structure having a defined shape and size.
41. The composition of paragraph 24, further comprising a solidifying agent for solidifying the microsphere, the at least one bioactive agent, and the at least one scaffold material into a fabricated structure having a defined shape and size.
42. The composition of paragraph 41, wherein the solidifying agent is alginate.
43. The composition of paragraph 40, wherein the solidified fabricated structure is porous.
44. The composition of paragraph 24, comprising a PLGA microsphere, VEGF, PDGF, peptide #237 (prom-1 #237), demineralized bone particles, and alginate, wherein the PLGA microsphere encapsulates PDGF within and is coated with VEGF externally.
45. The composition of paragraph 24, comprising a PLGA microsphere, PDGF, peptide #237 (prom-1 #237), demineralized bone particles, and alginate, wherein the PLGA microsphere encapsulates PDGF within.
46. The composition of paragraph 24, comprising a PLGA microsphere, peptide #237 (prom-1 #237), demineralized bone particles, and alginate.
47. The composition of paragraph 1 or 24, wherein the composition promotes bone growth and/or bone regeneration.
48. A method of promoting bone growth and/or bone regeneration in a hard tissue or an oral tissue defect, the method comprising contacting the tissue with a composition of paragraphs 1-47.
49. A method of treating or repairing a hard tissue defect or an oral tissue defect in a subject comprising contacting the defect with a composition of any one of paragraphs 1-47.
50. A method of treating or repairing a hard tissue defect or an oral tissue defect in a subject comprising selecting a subject having the defect and contacting the defect in the subject with a composition of any one of paragraphs 1-47.
51. The method of any one of paragraphs 48-50, wherein the hard tissue is selected from the group consisting of long bone, alveolar bone, cranial bone, facial bone, short bones, flat bones, irregular bones, sesamoid bones and cartilage.
52. The method of paragraph 49 or 50, wherein the oral tissue defect is selected from the group consisting of dentoalveolar defects, connective tissue defects, and collagen membrane defects.
53. The method of any one of paragraphs 48-50, wherein the composition is used during guided bone regeneration, connective tissue grafts, or sinus lift procedures.
54. The method of any one of paragraphs 48-50, wherein the composition is applied to a surgical implant or tissue graft which is implanted into the hard tissue or oral tissue of the subject

It should be understood that the compositions and methods described herein are not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages will mean±1%.

All patents and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

This invention is further illustrated by the following example which should not be construed as limiting. The contents of all references cited throughout this application, as well as the figures and table are incorporated herein by reference.

Those skilled in the art will recognize, or be able to ascertain using not more than routine experimentation, many equivalents to the specific embodiments of the invention described herein. Such equivalents are intended to be encompassed by the following claims.

### EXAMPLE

### Materials and Methods

Microspheres of PLGA were made by standard double-emulsion with PDGF added during this process. As a result, PDGF is encapsulated inside the microspheres. After lyophilizing the microspheres, the microspheres were mixed with other components for the scaffold preparation. VEGF was added at this point. The VEGF binds and coats to the outer surface of the microspheres. This powder mixture was then compressed and finalized by gas foaming technique. Leaching of the salt from this compressed mixture created the porous formation comprising the PLGA microspheres. This design allows the PLGA microspheres to release VEGF in a burst mode and release PDGF in a more sustained/prolonged timeframe. Also the porous nature of the scaffold structure (i.e. the PLGA microspheres) allows for cells migration on it. This scaffold was implanted over a critical size defect (e.g., 2 mm) created on the parietal bone of C57B1/6 mice. Skin was sutured over it. Animals are kept for 28 days after surgery, time in which the scaffold slowly degrades and releases the growth factors over bone/defect.

### Example 1

### Repair of Long Bone Defects

Improved repair of long bone defects is an area in which a particular need exists as such defects due to infection or trauma is particularly difficult to manage in human subjects. Because long bones are typically load-bearing structures, their repair, regeneration, and continued health is complicated and exposed to additional stresses not necessarily observed in other bone type defects. Long bone defects tend to affect subject's health and quality of life for at least several years and therefore improved methods of long bone tissue healing, repair, or regeneration is an ideal test of any new bone tissue repair method or composition.

The model animals used in this investigation were 10 week-old, skeletally mature, male C57/BI/6 wild-type mice. Mice are anesthetized by intramuscular injection with ketamine (7.5 mg/100 g body weight and xylazine (5-10 mg/kg body weight). The skin is shaved with an electric device and then scrubbed with a povidone-iodine scrub (e.g., Betadine®) and 70% alcohol, taking care to scrub from the center of the site toward the periphery. The site is then rinsed with 70% alcohol. At least three alternating preparations of germicidal scrub and rinse are performed. Finally, the area is draped with sterile drapes, which not only help to prevent contaminants from entering the surgical field, but also provide a sterile area on which to lay sterile instruments during surgery.

The surgeon thoroughly washes his/her hands with a bactericidal scrub and sterile surgical gloves are used. A surgical mask and clean gown are worn during surgery. To prevent corneal desiccation, bland ophthalmic ointment is placed in the eyes of the animals, following the onset of anesthesia.

Full-thickness flaps are utilized in order to gain access to the femur. A roughly 5 mm length segmental femur defect is created on the C57/BI/6 mice. Once the defect has been created all mice received cell-free grafts. Control mice received no additional treatment, while the second group of mice received treatment with prom-1 #237. Soft tissues are closed with sutures. After the surgery, the animals are monitored until fully alert and monitoring continues thereafter for any issues related to discomfort, pain or infection. Animals are given 10% dextrose solution with 268 ug/L ampicillin for one week post-op to maintain the energy while preventing infection. Medication to control pain is also delivered right after surgery and if detected subsequently. During recovery time, the animal is kept warm. Water-circulating heat pads are used for this purpose. The animal is monitored continuously until it regains sternal recumbence and is capable of holding its head up, and is returned to its regular cage only when it is alert, mobile and breathing normally. The animal is kept warm and dry and fluids, analgesics and antibiotics administered as required.

Post-surgical observations include a minimum daily observation, including weekends and holidays, of the condition of the animal and the surgical site. Animals are observed for continued recovery, which may include state of arousal; indices of pain of discomfort; condition of the surgical wound; appetite; hydration status; capillary refill time; mucous membrane color; or fecal and urine production. Pain/distress is monitored by monitoring the physiological parameters and behavioral responses although distress is more difficult to define and identify than pain. Physiological parameters to detect pain include hormonal responses (e.g., changes in the levels of adrenal hormones), increased susceptibility to disease (which may indicate an impaired immune system) or weight changes. The animals are evaluated at day 14 after the creation of the defect to determine the extent of repair and regeneration.

As depicted in FIG. 1A, the mice receiving only a cell-free graft (G) showed little evidence of new bone growth or repair of the defect. The graft is intact, but no new bone growth was detectable; there was not yet evidence of the defect being repaired. In contrast, mice receiving a prominin-1 #237 composition in addition to the cell-free graft, shown in FIG. 1B, exhibited significant callus formation (marked by the arrows), a sign that new bone was occurring and the defect was being healed, using the graft as a scaffold for bridging the defect. The addition of the bioactive agent (in this case, prom-1 #237) composition greatly increased the ability of the existing mouse cells to colonize the scaffold material and repair a large defect in the long bone. The total extent of recovery at this time point was markedly improved and the time to recovery was likely to be significantly reduced.

### Example 2

### Repair of Calvarial Bone Defect

One of the most common models used to test bone repair methods and compositions is defects in the calvarial bone in a model organism. Mice, rats, and rabbits are all frequently used in this model of flat bone repair. The model animals used in this investigation were 10 week-old, skeletally mature, male C57/BI/6 wild-type mice. Mice are anesthetized by intramuscular injection with ketamine (7.5 mg/100 g body weight and xylazine (5-10 mg/kg body weight). The calvaria skin is shaved with an electric device and then scrubbed with a povidone-iodine scrub (e.g., Betadine®) and 70% alcohol, taking care to scrub from the center of the site toward the periphery. The site is then rinsed with 70% alcohol. At least three alternating preparations of germicidal scrub and rinse are performed. Finally, the area is draped with sterile drapes, which not only help to prevent contaminants from entering the surgical field, but also provide a sterile area on which to lay sterile instruments during surgery.

The surgeon thoroughly washes his/her hands with a bactericidal scrub and sterile surgical gloves are used. A surgical mask and clean gown are worn during surgery. To prevent corneal desiccation, bland ophthalmic ointment is placed in the eyes of the animals, following the onset of anesthesia.

Full-thickness flaps are utilized in order to gain access to the calvaria. A roughly 2 mm diameter craniotomy defect is created on the C57/BI/6 mice using a dental trephine to contour and perforate the calvaria and subsequently remove the calvarial bone with the help of a periosteal elevator, attempting to avoid perforation of the dura-mater. Once the defect has been created, soft tissues are closed with sutures. After the surgery, the animals are monitored until fully alert and monitoring continues thereafter for any issues related to discomfort, pain or infection. Animals are given 10% dextrose solution with 268 ug/L ampicillin for one week post-op to maintain the energy while preventing infection. Medication to control pain is also delivered right after surgery and if detected subsequently. During recovery time, the animal is kept warm. Water-circulating heat pads are used for this purpose. The animal is monitored continuously until it regains sternal recumbence and is capable of holding its head up, and is returned to its regular cage only when it is alert, mobile and breathing normally. The animal is kept warm and dry and fluids, analgesics and antibiotics administered as required.

Post-surgical observations include a minimum daily observation, including weekends and holidays, of the condition of the animal and the surgical site. Animals are observed for continued recovery, which may include state of arousal; indices of pain of discomfort; condition of the surgical wound; appetite; hydration status; capillary refill time; mucous membrane color; or fecal and urine production. Pain/distress is monitored by monitoring the physiological parameters and behavioral responses although distress is more difficult to define and identify than pain. Physiological parameters to detect pain include hormonal responses (e.g., changes in the levels of adrenal hormones), increased susceptibility to disease (which may indicate an impaired immune system) or weight changes. The osseous defects are treated by injection with one of the various combinations of scaffold and growth factor. The experimental groups are (a) injections of saline, (b) injections of a freeze-dried bone allograft (FDBA) and saline composition and (c) injections of a FDBA and prom-1 #237 composition. The animals are evaluated at day 56 after the creation of the defect to determine the extent of repair and regeneration.

As depicted in FIG. 2, the mice receiving saline injections, represented by skull a, show little evidence of new bone growth or repair of the defect. The original defect is still clearly visible. Mice receiving FDBA and saline composition, represented by skull b, show evidence of a minor amount of repair. Some bone growth in the area of the defect is noticeable, but only a portion of the defect has been replaced by new bone and a large area of defect remains. In contrast, mice receiving FDBA and prom-1 #237 composition, represented by skull c, show complete replacement of the bone defect area. No remaining defect is visible and a large area of de novo bone growth is apparent.

The addition of the bioactive agent (in this case, prom-1 #237) to the composition containing FDBA greatly increases the ability of the existing mouse cells to colonize the FDBA scaffold material and repair a large defect in the calvarial flat bone. The total extent of recovery is markedly improved and the time to recovery is significantly reduced.

### Example 3

### Repair of Flat Bones Using Microsphere Compositions

The model animals used in this investigation are 10 week-old, skeletally mature, male Sprague-Dawley rats, a model previously established by (Schmitz & Hollinger, Clin Orthop Relat Res 1986 205:299-208; Intini et al., J Periodontol 2008 79:1217-24). Rats are anesthetized by intramuscular injection with ketamine (7.5 mg/100 g body weight and xylazine (5-10 mg/kg body weight). The calvaria skin is shaved with an electric device and then scrubbed with a povidone-iodine scrub (e.g. Betadine®) and 70% alcohol, taking care to scrub from the center of the site toward the periphery. The site is then rinsed with 70% alcohol. At least three alternating preparations of germicidal scrub and rinse are performed. Finally, the area is draped with sterile drapes, which not only help to prevent contaminants from entering the surgical field, but also provide a sterile area on which to lay sterile instruments during surgery.

The surgeon thoroughly washes his/her hands with a bactericidal scrub and sterile surgical gloves are used. A surgical mask and clean gown are worn during surgery. To prevent corneal desiccation, bland ophthalmic ointment is placed in the eyes of the animals, following the onset of anesthesia.

Full-thickness flaps are utilized in order to gain access to the calvaria. A roughly 8 mm diameter craniotomy defect is created on the Sprague-Dawley rats using a dental trephine to contour and perforate the calvaria and subsequently remove the calvarial bone with the help of a periosteal elevator, attempting to avoid perforation of the dura-mater. Once the defect has been created, soft tissues are closed with sutures. After the surgery, the animals are monitored until fully alert and monitoring continues thereafter for any issues related to discomfort, pain or infection. Animals are given 10% dextrose solution with 268 ug/L ampicillin for one week post-op to maintain the energy while preventing infection. Medication to control pain is also delivered right after surgery and if detected subsequently. During recovery time, the animal is kept warm. Water-circulating heat pads are used for this purpose. The animal is monitored continuously until it regains sternal recumbence and is capable of holding its head up, and is returned to its regular cage only when it is alert, mobile and breathing normally. The animal is kept warm and dry and fluids, analgesics and antibiotics administered as required.

Post-surgical observations include a minimum daily observation, including weekends and holidays, of the condition of the animal and the surgical site. Animals are observed for continued recovery, which may include state of arousal; indices of pain of discomfort; condition of the surgical wound; appetite; hydration status; capillary refill time; mucous membrane color; or fecal and urine production. Pain/distress is monitored by monitoring the physiological parameters and behavioral responses although distress is more difficult to define and identify than pain. Physiological parameters to detect pain include hormonal responses (e.g., changes in the levels of adrenal hormones), increased susceptibility to disease (which may indicate an impaired immune system) or weight changes. The animals are evaluated at day 56 after the creation of the defect to determine the extent of repair and regeneration. The 8 treatments will be applied to 8 groups of 5 rats each (estimating a 90% power and type I error=0.05). All animals in all groups will have the calvaria defect created and the treatment composition placed under a dome membrane, a Millipore filter (pore size 0.22 μg) that had been cut and fabricated by means of cyanoacrylate medical-grade adhesive cement, as a dome shape (8 mm diameter×2 mm height) (Kimoto et al., J Dent Res. 1998 13852593.4 77:1965-9). The treatment protocols are as follows: group 1: FDBA scaffold with PDGF-BB and VEGF incorporated PLGA microspheres; group 2: FDBA scaffold with PDGF-BB incorporated PLGA microspheres; group 3: FDBA with VEGF incorporated PLGA microspheres; group 4: FDBA with PLGA-microspheres with no bioactive agents; group 5: only FDBA scaffold; group 6: no filling biomaterials (negative control); group 7: FDBA with PLGA-microspheres with Prominin-1 #237; group 8: FDBA with PLGA-microspheres containing the Prominin-1 #237 and microspheres containing VEGF. The endogenous VEGF and PDGF effect on bone healing will be evaluated through group 5 & 6 and compared to the exogenous growth factors effect associated to the natural expression of those factors present on the other groups, simulating a clinical application of these biomaterials.

A bone fluorochrome (calcein, 15 mg/kg injected IM) will be administered 1-day post surgery and every 3^{rd} day to demarcate the region between old and new bone formation (Rohrer and Schubert, Oral Surg Oral Med Oral Pathol 1992 74; 73-8). Before use, calcein is adjusted to pH value of 7.2 and sterilized by filtration.

The animals will be evaluated until day 56 after surgery, then all animals will be sacrificed using CO₂ followed by bilateral thoracotomy euthanasia. Craniotomy sites with 10 mm contiguous bone will be recovered from the skull and will be processed for histological analysis as previously described (Rohrer and Schubert, Oral Surg Oral Med Oral Pathol 1992 74; 73-8). Block biopsies will be harvested, then fixed and stored in 10% formalin before initial sectioning, what is done by a cutting-grinding system. After sectioning, specimen will be fixed in 10% formalin for 1 day, and then will be dehydrated with successive alcohol or GMA (2-hydroxyethyl methacrylate) concentrations. Plastic infiltration will be achieved with a 1:1 combination of GMA and embedding medium, followed by immersions in 100% embedding medium. A two-step photopolymerization process is carried out in an apparatus with two white lights of 450 nm wavelength. In the first step, a gradual polymerization is carried for 2 hours and the second occurs over 4 hours under blue lights. Finally, a microgrinding system is applied to prepare the slide sections. Each section will be made to produce 7 μm thick sections. 2 sections of each animal will be analyzed, both corresponding to the center of the craniotomy. Toluidine blue, a metachromatic stain, will be used for the histological staining as it's highly recommended due its easy application and clear definition of bone apposition and resorption.

To test the ability of PDGF-BB/VEGF polymer microsphere system to regenerate bone and promote neo-vascularization, the microstructure of the engineered bone tissue formed will be analyzed using histologically stained samples in a histomorphometrical analysis, comparing new bone formation and its characteristics, including cell counts. A comparison will be made between the groups. To evaluate the calcein, a fluorescent microscope with XBO 75 xenon short-arc lamp and appropriate fluorescence filter sets will be used. Fluorescence microscopic images will be acquired using a Sagnac type interferometer as well as a conventional digital camera with a fluorescence mode as control (Pautke et al., Bone 2005 37:441-5). Radiographs will also be taken to compare density of calcification.

Two blinded examiners will review the slides. Any slides that showed processing errors that would alter measurements would be excluded. The histological review will serve to evaluate the spatial and temporal sequence of osteogenesis during healing. Analysis of standard parameters in osteogenesis will be completed and each group will be compared to each other using the unpaired t-test.

### Example 4

### Repair of Long Bones Using Microsphere Compositions

The model animals used in this investigation are 10 week-old, skeletally mature, male Sprague-Dawley rats, a model previously established by (Schmitz & Hollinger, Clin Orthop Relat Res 1986 205:299-208; Intini et al., J Periodontol 2008 79:1217-24). Mice are anesthetized by intramuscular injection with ketamine (7.5 mg/100 g body weight and xylazine (5-10 mg/kg body weight). The calvaria skin is shaved with an electric device and then scrubbed with a povidone-iodine scrub (e.g. Betadine®) and 70% alcohol, taking care to scrub from the center of the site toward the periphery. The site is then rinsed with 70% alcohol. At least three alternating preparations of germicidal scrub and rinse are performed. Finally, the area is draped with sterile drapes, which not only help to prevent contaminants from entering the surgical field, but also provide a sterile area on which to lay sterile instruments during surgery.

Sprague-Dawley rats, a model previously established by (Schmitz & Hollinger, Clin Orthop Relat Res 1986 205:299-208; Intini et al., J Periodontol 2008 79:1217-24). Rats are anesthetized by intramuscular injection with ketamine (7.5 mg/100 g body weight and xylazine (5-10 mg/kg body weight). The skin is shaved with an electric device and then scrubbed with a povidone-iodine scrub (e.g. Betadine®) and 70% alcohol, taking care to scrub from the center of the site toward the periphery. The site is then rinsed with 70% alcohol. At least three alternating preparations of germicidal scrub and rinse are performed. Finally, the area is draped with sterile drapes, which not only help to prevent contaminants from entering the surgical field, but also provide a sterile area on which to lay sterile instruments during surgery.

The surgeon thoroughly washes his/her hands with a bactericidal scrub and sterile surgical gloves are used. A surgical mask and clean gown are worn during surgery. To prevent corneal desiccation, bland ophthalmic ointment is placed in the eyes of the animals, following the onset of anesthesia.

Full-thickness flaps are utilized in order to gain access to the femur. A roughly 5 mm length segmental femur defect is created on the Sprague-Dawley rats. Once the defect has been created all rats receive cell-free grafts and an additional treatment detailed below. Soft tissues are closed with sutures. After the surgery, the animals are monitored until fully alert and monitoring continues thereafter for any issues related to discomfort, pain or infection. Animals are given 10% dextrose solution with 268 ug/L ampicillin for one week post-op to maintain the energy while preventing infection. Medication to control pain is also delivered right after surgery and if detected subsequently. During recovery time, the animal is kept warm. Water-circulating heat pads are used for this purpose. The animal is monitored continuously until it regains sternal recumbence and is capable of holding its head up, and is returned to its regular cage only when it is alert, mobile and breathing normally. The animal is kept warm and dry and fluids, analgesics and antibiotics administered as required.

Post-surgical observations include a minimum daily observation, including weekends and holidays, of the condition of the animal and the surgical site. Animals are observed for continued recovery, which may include state of arousal; indices of pain of discomfort; condition of the surgical wound; appetite; hydration status; capillary refill time; mucous membrane color; or fecal and urine production. Pain/distress is monitored by monitoring the physiological parameters and behavioral responses although distress is more difficult to define and identify than pain. Physiological parameters to detect pain include hormonal responses (e.g., changes in the levels of adrenal hormones), increased susceptibility to disease (which may indicate an impaired immune system) or weight changes. The animals are evaluated at day 14 after the creation of the defect to determine the extent of repair and regeneration.

The 8 treatments will be applied to 8 groups of 5 rats each (estimating a 90% power and type I error=0.05). All animals in all groups will have the long bone defect created and one of the following treatment protocols: group 1: FDBA scaffold with PDGF-BB and VEGF incorporated PLGA microspheres; group 2: FDBA scaffold with PDGF-BB incorporated PLGA microspheres; group 3: FDBA with VEGF incorporated PLGA microspheres; group 4: FDBA with PLGA-microspheres with no bioactive agents; group 5: only FDBA scaffold; group 6: no filling biomaterials (negative control); group 7: FDBA with PLGA-microspheres with Prominin-1 #237; group 8: FDBA with PLGA-microspheres containing the Prominin-1 #237 and microspheres containing VEGF. The endogenous VEGF and PDGF effect on bone healing will be evaluated through group 5 & 6 and compared to the exogenous growth factors effect associated to the natural expression of those factors present on the other groups, simulating a clinical application of these biomaterials.

A bone fluorochrome (calcein, 15 mg/kg injected IM) will be administered 1-day post surgery and every 3^{rd} day to demarcate the region between old and new bone formation (Rohrer and Schubert, Oral Surg Oral Med Oral Pathol 1992 74; 73-8). Before use, calcein is adjusted to pH value of 7.2 and sterilized by filtration.

The animals will be evaluated until day 14 after surgery, then all animals will be sacrificed using CO₂ followed by bilateral thoracotomy euthanasia. Femurs will be recovered and will be processed for histological analysis as previously described (Rohrer and Schubert, Oral Surg Oral Med Oral Pathol 1992 74; 73-8). Block biopsies will be harvested, then fixed and stored in 10% formalin before initial sectioning, what is done by a cutting-grinding system. After sectioning, specimen will be fixed in 10% formalin for 1 day, and then will be dehydrated with successive alcohol or GMA (2-hydroxyethyl methacrylate) concentrations. Plastic infiltration will be achieved with a 1:1 combination of GMA and embedding medium, followed by immersions in 100% embedding medium. A two-step photopolymerization process is carried out in an apparatus with two white lights of 450 nm wavelength. In the first step, a gradual polymerization is carried for 2 hours and the second occurs over 4 hours under blue lights. Finally, a microgrinding system is applied to prepare the slide sections. Each section will be made to produce 7 μm thick sections. 2 sections of each animal will be analyzed, both corresponding to the center of the defect. Toluidine blue, a metachromatic stain, will be used for the histological staining as it's highly recommended due its easy application and clear definition of bone apposition and resorption.

To test the ability of PDGF-BB/VEGF polymer microsphere system to regenerate bone and promote neo-vascularization, the microstructure of the engineered bone tissue formed will be analyzed using histologically stained samples in a histomorphometrical analysis, comparing new bone formation and its characteristics, including cell counts. A comparison will be made between the groups. To evaluate the calcein, a fluorescent microscope with XBO 75 xenon short-arc lamp and appropriate fluorescence filter sets will be used. Fluorescence microscopic images will be acquired using a Sagnac type interferometer as well as a conventional digital camera with a fluorescence mode as control (Pautke et al., Bone 2005 37:441-5). Radiographs will also be taken to compare density of calcification.

Two blinded examiners will review the slides. Any slides that showed processing errors that would alter measurements would be excluded. The histological review will serve to evaluate the spatial and temporal sequence of osteogenesis during healing. Analysis of standard parameters in osteogenesis will be completed and each group will be compared to each other using the unpaired t-test.

The references cited herein and throughout the specification are incorporated herein by reference in their entirety.

What is claimed:

1. A composition for promoting bone growth or bone regeneration comprising at least one bioactive agent, a first scaffold material, and at least one other scaffold material.

2. The composition of claim 1, wherein the at least one bioactive agent is an agent that promotes angiogenesis or an agent that promotes bone growth.

3. The composition of claim 2, wherein the at least one bioactive agent is selected from the group consisting of:
vascular endothelial growth factor (VEGF), platelet-derived growth factor (PDGF), fibroblast growth factor (FGF); prominin-1 peptide (peptide #237); bone morphogenetic protein 2 (BMP2); bone morphogenic protein 4 (BMP4); and bone morphogenic protein 7 (BMP7).

4. The composition of claim 1, wherein the first scaffold material is bone fragments or bone-derived fragments.

5. The composition of claim 1, wherein the at least one other scaffold material is selected from the group consisting of:
glycosaminoglycan, silk, fibrin, MATRIGEL™, poly-ethyleneglycol (PEG), polyhydroxy ethyl methacrylate, polyvinyl alcohol, polyacrylamide, poly(N-vinyl pyrolidone), poly glycolic acid (PGA), poly lactic-co-glycolic acid (PLGA), poly e-carpolactone (PCL), polyethylene oxide, poly propylene fumarate (PPF), poly acrylic acid (PAA), hydrolysed polyacrylonitrile, polymethacrylic acid, polyethylene amine, alginic acid, pectinic acid, carboxy methyl cellulose, hyaluronic acid, heparin, heparin sulfate, chitosan, carboxymethyl chitosan, chitin, pullulan, gellan, xanthan, collagen, gelatin, carboxymethyl starch, carboxymethyl dextran, chondroitin sulfate, cationic guar, and cationic starch as well as salts and esters thereof.

6. The composition of claim 1, comprising PLGA, peptide #237 (prom-1 #237), demineralized bone particles and optionally, VEGF and/or PDGF.

7. The composition of claim 1, wherein the at least one bioactive agent, the first scaffold material, and the at least one other scaffold material are solidified together into a fabricated structure having a defined shape and size.

8. The composition of claim 7, further comprising a solidifying agent.

9. The composition of claim 8, wherein the solidifying agent is alginate.

10. The composition of claim 7, wherein the solidified fabricated structure is porous.

11. The composition of claim 1, wherein the at least one other scaffold material forms a microsphere.

12. The composition of claim 11, wherein the microsphere comprises the at least one bioactive agent.

13. The composition of claim 11, wherein the microsphere is a PLGA microsphere.

14. The composition of claim 13, comprising a PLGA microsphere, peptide #237 (prom-1 #237), demineralized bone particles, and optionally, VEGF and/or PDGF.

15. The composition of claim 14, wherein the PLGA microsphere encapsulates PDGF and is coated with VEGF externally.

16. A method of treating or repairing a hard tissue defect or an oral tissue defect in a subject comprising contacting the defect with a composition of claim 1.

17. The method of claim 16, further comprising selecting a subject having a hard tissue or oral tissue defect.

18. The method of claim 16, wherein the defect is selected from the group consisting of:
long bone defects; alveolar bone defects; cranial bone defects; facial bone defects; short bone defects; flat bone defects; irregular bone defects; sesamoid bone defects; cartilage defects; dentoalveolar defects; connective tissue defects; and collagen membrane defects.

19. The method of claim 16, wherein the composition is used during guided bone regeneration, connective tissue grafts, or sinus lift procedures.

20. The method of claim 16, wherein the composition is applied to a surgical implant or tissue graft which is implanted into the hard tissue or oral tissue of the subject.
